(19) United States
(12) Patent Application Publication
(10) Pub. No.: US 2024/0024372 A1
(43) Pub. Date: Jan. 25, 2024

(54) COMPOUNDS AND METHODS TO TREAT CYSTIC FIBROSIS

(71) Applicant: City of Hope, Duarte, CA (US)

(72) Inventors: Kevin V. Morris, Duarte, CA (US); Olga Raquel Villamizar Beltran, Duarte, CA (US)

(21) Appl. No.: 18/265,544

(22) PCT Filed: Dec. 6, 2021
(86) PCT No.: PCT/US2021/061963

§ 371 Date: Jun. 6, 2023

Related U.S. Application Data
(60) Provisional application No. 63/123,413, filed on Dec. 9, 2020.

(51) Int. Cl. A61K 35/42; C07K 14/47

(52) U.S. Cl. CPC A61K 35/42; C07K 14/4712; C07K 2319/81; C07K 2319/09; C07K 2319/71

(57) ABSTRACT

The disclosure provides, inter alia, fusion proteins comprising a zinc finger domain and a transcriptional activator, nucleic acids, vectors, and exosomes that can be used to activate transcription in a cystic fibrosis transmembrane conductance regulator gene and treat cystic fibrosis.

### CROSS-REFERENCE TO RELATED APPLICATIONS

This application claims priority to U.S. Application No. 63/123,413 filed Dec. 9, 2020, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant no. DK104681-02 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-789001WO_ST25.txt, created Nov. 16, 2021, 68,361 bytes, machine format IBM-PC, MS Windows operating system, is hereby incorporated by reference.

### BACKGROUND

Cystic fibrosis (CF) is an autosomal recessive genetic disease caused by mutations in the CFTR gene (1). The CFTR protein is a c-AMP activated chloride ion channel present in many organs including the lungs, pancreas, and reproductive glands (2). Mutations in the CFTR gene result in a frameshift mutation in the mRNA coding sequence that impinges protein processing, channel gating, conductance function and ultimately results in the expression of a relatively non-functional CFTR protein. The most prevalent CFTR mutation is caused by the deletion of a phenylalanine residue at position 508 (F508del) in the CFTR protein, which affects about 70% of patients (3). A small proportion of this misfolded F508del protein is however released from the Golgi and presented on the plasma membrane with a reduction in channel gating activity (4-6). There are a plethora of other CFTR mutations (7), which ultimately result in diminished CFTR protein function, and simply increasing its expression could improve the CF phenotype. A means to target and activate CFTR, irrespective of the variation in CFTR mutations, could prove therapeutically relevant in CF patients.

Novel targeted therapeutic approaches have emerged for the treatment of CF using small molecule modulators to enhance or restore the functional expression of mutated CFTR (8). CFTR modulators ivacaftor, lumacaftor/ivacaftor, tezacaftor/ivacaftor, and elexacaftor/tezacaftor/ivacaftor have been approved by the U.S. Food and Drug Administration for those people with specific CF mutations (9). Treatment with more than one CFTR modulator appears to be the most optimal strategy for many CFTR mutations, to improve CFTR function by enhancing cellular chaperone function VX-809 (lumacaftor) and acting as potentiator that increases channel gating and conductance VX-770 (ivacaftor) (10).

While this new line of therapeutics improves the current standard of care for a subset of CF patients, it is only beneficial for those individuals with eligible CFTR mutations. A method that could result in a sustained increase in cell surface expression that is recalcitrant to the various mutant forms of CFTR could prove clinically impactful for those individuals who are refractory to the current modulator therapy. The disclosure is directed to this, as well as other, important ends.

### BRIEF SUMMARY

Provided herein are fusion proteins comprising a zinc finger domain and a transcriptional activator. The fusion proteins are capable of binding to a cystic fibrosis transmembrane conductance regulator gene. Also provided herein are nucleic acids encoding the fusion proteins; and vectors having nucleic acids encoding the fusion proteins.

Provided herein are exosomes comprising the fusion proteins, nucleic acids, or vectors described herein. The exosomes can be mesenchymal stem cell exosomes. In embodiments, the disclosure provides pharmaceutical compositions comprising the exosomes described herein.

Provided herein are methods of treating cystic fibrosis by activating transcription in a cystic fibrosis transmembrane conductance regulator gene using the pharmaceutical compositions, exosomes, vectors, nucleic acids, and fusion proteins described herein.

These and other embodiments of the disclosure are provided in more detail herein.

### BRIEF DESCRIPTION OF THE DRAWINGS

FIGS. 1A-1E show CFTR gene activation using Zinc Finger activator protein of SEQ ID NO:3. FIG. 1A: A schematic of the zinc finger binding domain targeted by CFZF in the CFTR promoter. The target sequence is shown along with its genomic location as determined from the UCSC genome browser. FIG. 1B: Schematic of vector expressing the FLAG-CFZF-VPR. A CMV Pol II promoter expresses a FLAG-tagged CFZF (SEQ ID NO:2) targeted to the CFTR promoter with a VPR activation domain. FIG. 1C ChIP assays were performed and enrichment relative to control (Neg) is shown as a fraction of input. FIG. 1D: CFTR mRNA levels as determined by qRT-PCR in HuBECs from healthy donor (HuBEC CFTR-WT). FIG. 1E: HuBECs from CF patient with the F508del mutation (HuBEC CFTR-F508del). HuBEC non-transfected were used as negative control (Neg control). Experiments show the standard error of the mean and p values are represented (paired two-sided Student's T-test, *p=0.01, **p<0.02).

FIGS. 2A-2F show that MSC exosome mediated delivery of Zinc Finger Protein Activator of SEQ ID NO:3 increases the expression of CFTR. FIG. 2A: TEM micrograph of exosomes isolated from the culture medium of MSC-CFZF-VPR transfected cells. Exosomes were measured by using Nanosight NS 300 system in the supernatant from cultures cells. The histogram represents particle size distribution. FIG. 2B: Western blot analysis for exosome markers in CFZF-VPR transfected MSC (CFZF) and non-transfected MSCs (Neg) derived exosomes. FIG. 2C: Western blot of FLAG-tagged CFZF-VPR protein enriched in exosomes from left: MSC-CFZF-VPR or right: non-transfected MSC-exosomes (M-Ex), HEK293-exosomes (H-Ex) and corresponding MSC cell lysate (M-CL) samples, respectively. FIG. 2D: Evaluation by qRT-PCR of mRNA expression of exosomes from CFZF-VPR/Cx43-transfected MSCs. The results from triplicate exosomes collected from three different CFZF-VPR/Cx43 transfected MSCs are shown. FIG. 2E: Light microscopy immunofluorescence images of HuBECs uptake of MSCs-CFZF-VPR exosomes labeled with BODIPY TR ceramide, Nuclei, Actin. Scale bar, 10 μm. FIG. 2F: CFTR expression was determined by qRT-PCR following treatment with MSC exosomes directed to the CFTR promoter (MSC-CFZF-VPR) or Control (MSC-Neg) in HuBECs. For E and F experiments were performed in triplicate with 10e+03 HuBECs treated with 5e+10 exosomes. Experiment shown the standard error of the means and p values from a paired two-sided T-test, *p=0.01

FIGS. 3A-3E show that exosome mediated delivery of CFZF-VPR (SEQ ID NO:3) increases CFTR protein expression and enhanced Chloride transport. CFTR ELISA analysis of CFTR protein expression in (FIG. 3A) HuBECs from healthy donor HuBEC-WT and (FIG. 3B) in patient cells with F508del mutation following transfection with CFZF-VPR plasmid or treated with MSC-derived exosomes carrying CFZF-VPR. FIG. 3C: A schematic is shown depicting the halide assay used to assess chloride transport. Fluorescence decrease was evaluated at 0, 2 and 60 seconds in response to exchange of 25 mM of sodium iodide in cells treated with 0.3 μM of forskolin and combination of VX-809 and VX-770, current drugs used for treatment of CF patients. FIG. 3D HuBEC CFTR-WT and FIG. 3E HuBEC F508del cells were transfected with CFZF-VPR plasmid or treated with MSC-derived exosomes carrying CFZF-VPR and assessed 48 hours post-treatment using the halide assay. Experiments were performed in triplicate with 10e+03 HuBEC treated with 5e+10 exosomes. ELISA and halide experiments were performed in triplicate in cells shown with the standard error of the means and p values from a paired two-sided T-test, *p<0.05.

FIG. 4 shows the underlying mechanism of MSC-CFZF-VRP (e.g., SEQ ID NO 2) exosome mediated activation CFTR. MSC-exosome are generated to contain a CFZF-VPR targeted to the CFTR promoter and are internalized into human bronchial epithelial cells whereby the CFZF-VPR protein and CFZF-VPR mRNA are released into the intracellular environment by the action of Cx43 and presumably interactions with other endogenous exosome pathway proteins. The result of this Cx43 mediated delivery of CFZF-VPR is increased expression and functional activation of CFTR.

FIGS. 5A-5G show that HEK293 exosome mediated delivery of CFZF-VPR (SEQ ID NO:3) increases expression of CFTR. FIG. 5A: TEM micrographs of CFZF-VPR containing exosomes isolated from the culture medium of HEK293 cells. Exosomes were measured by using Nanosight NS 300 system in the supernatant from cultures cells. The histogram represents particle size distribution. FIGS. 5B-5C: Western blot analysis FIG. 5B for exosome markers in HEK293-derived exosomes from non-transfected HEK293 cells (Neg) and HEK293 cells transfected with (CFZF-VPR). Flag-tagged CFZF-VPR protein (SEQ ID NO:2) enriched in exosomes from CFZF-transfected HEK293. HuBEC cells are also shown FIG. 5C CFZF-VPR-flag tagged protein from WT HuBEC non-exosome treated (Neg) cells and WT and F508del HuBECs treated with MSC-derived exosomes loaded with CFZF-VPR. FIG. 5D: CFTR mRNA expression as determined by qRT-PCR from CFZF-VPR transfected (HEK-CFZF-VPR) and control (293-Neg) HEK293 cells. FIG. 5E: Light microscopy immunofluorescence images of HuBECs as negative control (no-exosomes) for uptake with BODIPY TR ceramide (red), Nuclei (Blue) Actin (Green), Scale bar. 10 μm. FIG. 5F: CFTR transcript expression after treatment with HEK293-derived exosomes directed to the CFTR promoter (HEK-CFZF-VPR), Control (293-Neg) in F508del and FIG. 5G CFTR transcript expression after treatment with MSC-derived exosomes directed to the CFTR promoter (MSC-CFZF-VPR) in CFTR-HuBEC WT cells. Experiments were performed in triplicate with 10e+03 HuBEC treated with 5e+10 exosomes. Experiment shown the standard error of the means and p values from a paired two-sided T-test, *p=0.01.

FIGS. 6A-6B show that HEK293 exosome mediated delivery of CFZF-VPR (SEQ ID NO:3) enhances CFTR Chloride transport. FIG. 6A: CFTR ELISA showing CFTR protein levels expression increased in HuBECs from CF patients with F508del mutation after treatment with HEK293 CFZF-VPR packaged exosomes (HEK-CFZF-VPR) relative to HEK293 cell control exosome treatment (HEK-Neg). FIG. 6B: CFTR-mediated Halide transport in EYFP showed fluorescence decrease in F508del HUBECs treated with HEK293-exosomes containing CFZF-VPR relative to control HEK293 exosomes (HEK-Neg).

FIGS. 7A-7B show exosome uptake inhibition. FIG. 7A: To study the pathway of exosome uptake, HuBEC cells were pre-treated with 200 μM of Genistein and incubated at 37° C. for 30 minutes before exosome addition, and then incubated with BODIPY-labeled exosomes at 37° C. for 3 h. FIG. 7B: Uptake was quantified by determining the percentage of BODIPY-fluorescence intensity.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices, and materials similar or equivalent to those described herein can be used. The following definitions are provided to facilitate understanding of certain terms used frequently herein and are not meant to limit the scope of the present disclosure.

The term “CFTR” or “cystic fibrosis transmembrane conductance regulator” refers to and includes any of the recombinant or naturally-occurring forms of CFTR or variants or homologs thereof that maintain CFTR activity (e.g. within at least 50%, 80%, 90%, 95%, 96%, 97%, 98%, 99% or 100% activity compared to CFTR). In embodiments, the variants or homologs have at least 90%, 95%, 96%, 97%, 98%, 99% or 100% amino acid sequence identity across the whole sequence or a portion of the sequence compared to a naturally occurring CFTR. In embodiments, CFTR is identified by the UniProt reference number P13569, or a variant, homolog or functional fragment thereof. The CFTR protein is a c-AMP activated chloride ion channel present in many organs, including the lungs, pancreas, and reproductive glands. Mutations in the CFTR gene lead to defects in frameshift of mRNA, protein processing, channel gating, conductance function and expression of non-functional CFTR protein, resulting in the production of altered mucus that is exceedingly viscous in the context of respiratory epithelium. The most prevalent mutation is caused by the deletion of phenylalanine residue at position 508 (F508del) of the CFTR protein, which affects more than 70% of patients with cystic fibrosis. This misfolded F508del protein results in proteolytic degradation at the endoplasmic reticulum, however, a small proportion of this protein is released to the plasma membrane displaying a reduction in channel gating activity.

The term “CFTR promoter” or “cystic fibrosis transmembrane conductance regulator promoter” refers to the promoter of the CFTR. In embodiments, the CFTR promoter comprises SEQ ID NO:1.

The term “mesenchymal stem cell” or “MSC” are multipotent stromal cells that originate from the mesoderm of many tissues, including bone marrow, liver, spleen, peripheral blood, adipose, placenta, and umbilical cord blood, and have the capacity to self-renew and the ability to generate differentiated cells. MSCs release extracellular vesicles, including exosomes (i.e., MSC exosomes). Similar to exosomes from other cells types, MSC exosomes participate in intercellular communication and carry proteins, mRNA, and miRNA, into targeted cells. MSC exosomes are ideal vehicles to carry and deliver molecules to targeted cells including therapeutic genes, drugs, enzymes, or RNA. Mesenchymal stem cells are generally positive for the markers CD105, CD106, CD90, and CD73.

The term “extracellular vesicle” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and cultured cells.

The term “exosome” refers to a cell-derived small (between 20-300 nm in diameter) vesicles that are generated inside multivesicular bodies (MVBs) and are secreted when these compartments fuse with a plasma membrane. Upon the fusion of MVBs with the plasma membrane, exosomes are released into the extracellular environment and can be either taken up by target cells residing in the microenvironment or carried to distant sites via biological fluids. Exosomes are enriched in many bioactive molecules such as lipids, proteins, mRNAs, transfer RNA (tRNA), long noncoding RNAs (lncRNAs), microRNAs (miRNAs) and mitochondrial DNA (mtDNA). Most exosomes have an evolutionarily conserved set of proteins including tetraspanins (CD81, CD63, and CD9), heat-shock proteins (HSP60, HSP70 and HSP90), ALIX and tumor susceptibility gene 101 (TSG101); however, they also have unique tissue type-specific proteins that reflect their cellular sources. Exosomes comprise a membrane that encloses an internal space, and which is generated from the cell by direct plasma membrane budding or by fusion of the late endosome with the plasma membrane. The exosome comprises lipid and/or fatty acid and optionally comprises a payload (e.g., a therapeutic agent), a receiver (e.g., a targeting peptide), a polynucleotide (e.g., a nucleic acid, RNA, or DNA), a sugar (e.g., a simple sugar, polysaccharide, or glycan) or other molecules or drugs.

The term “zinc finger domain” refers to a protein, or a domain within a larger protein, that binds DNA in a sequence-specific manner through one or more zinc fingers, which are regions of amino acid sequence within the binding domain whose structure is stabilized through coordination of a zinc ion. In embodiments, the zinc finger domain is non-naturally occurring in that it is engineered to bind to a target site of choice. In embodiments, a zinc finger domain has a sequence of the form X₃-Cys-X₂₋₄-Cys-X₁₂-His-X₃₋₅-His-X₄, wherein X is any amino acid (e.g., X₂₋₄ indicates an oligopeptide 2-4 amino acids in length). There is generally a wide range of sequence variation in the 28-31 amino acids of the known zinc finger domains. Only the two consensus histidine residues and two consensus cysteine residues bound to the central zinc atom are invariant. Of the remaining residues, three to five are highly conserved, while there may be significant variation among the other residues. Despite the wide range of sequence variation in the protein, zinc fingers of this type have a similar three dimensional structure. However, there is a wide range of binding specificities among the different zinc finger domains, i.e., different zinc fingers bind double stranded polynucleotides having a wide range of nucleotides sequences. In embodiments, the zinc finger domain is the C2H2 type. In embodiments, the zinc finger domain is the CCHC type. In embodiments, the zinc finger domain is the PHD type. In embodiments, the zinc finger domain is the RING type. In embodiments, the zinc finger domain includes the amino acid sequence of SEQ ID NO:8. In embodiments, the zinc finger domain has the amino acid sequence of SEQ ID NO:8. In embodiments, the zinc finger domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:8.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “transcriptional activator” refers to a protein that increases gene transcription of a gene or set of genes. For example, transcriptional activators may be DNA-binding proteins that bind to enhancers or promoter-proximal elements. The transcriptional activators used in the fusion proteins described herein include, but are not limited to, viral protein P, p65 transactivating subunit of NF-kappa B (p65), heat-shock factor 1 activation domain, VP16 activation domain, VP64 activation domain (VP64), synergistic activation mediator, replication and transcription activator (Rta), and any derivatives thereof. In embodiments, the transcriptional activator is VP64, p65, Rta, or a combination of two or more thereof. In embodiments, the transcriptional activator may increase gene transcription of a gene or a set of genes that was/were previously silenced. Transcriptional activators and uses thereof may be found, for example, in Tanenbaum et al., A Protein-Tagging System for Signal Amplification in Gene Expression and Fluorescence Imaging. Cell. 2014 Oct. 23:159(3):635-46 and Zalatan et al., Engineering Complex Synthetic Transcriptional Programs With CRISPR RNA Scaffolds. Cell. 2015 Jan. 15; 160(1-2):339-50.

The term “VP64” or “VP64 protein” or “VP64 activation domain” as provided herein includes any of the recombinant or naturally-occurring forms of tegument protein VP16 (VP64), also known as alpha trans-inducing protein, alpha-TIF, or variants or homologs thereof that maintain VP64 protein activity (e.g. within at least 50%, 80%, 90%, 95%, 96%, 97%, 98%, 99% or 100% activity compared to VP64 protein). In embodiments, the variants or homologs have at least 90%, 95%, 96%, 97%, 98%, 99% or 100% amino acid sequence identity across the whole sequence or a portion of the sequence (e.g., a 5, 10, 15, or 20 continuous amino acid portion) compared to a naturally occurring VP64 protein. In embodiments, VP64 protein is identified by the UniProt reference number P06492, or a variant, homolog or functional fragment thereof. In embodiments, VP64 includes the amino acid sequence of SEQ ID NO:9. In embodiments, VP64 has the amino acid sequence of SEQ ID NO:9. In embodiments, VP64 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:9. In embodiments, VP64 has an amino acid sequence that has at least 85% sequence identity to SEQ ID NO:9. In embodiments, VP64 has an amino acid sequence that has at least 90% sequence identity to SEQ ID NO:9. In embodiments, VP64 has an amino acid sequence that has at least 92% sequence identity to SEQ ID NO:9. In embodiments, VP64 has an amino acid sequence that has at least 94% sequence identity to SEQ ID NO:9. In embodiments, VP64 has an amino acid sequence that has at least 95% sequence identity to SEQ ID NO:9. In embodiments, VP64 has an amino acid sequence that has at least 96% sequence identity to SEQ ID NO:9. In embodiments, VP64 has an amino acid sequence that has at least 98% sequence identity to SEQ ID NO:9.

The term “p65” or “p65 protein” as provided herein includes any of the recombinant or naturally-occurring forms of Transcription factor p65 (p65), also known as nuclear factor NF-kappa-B p65 subunit, or variants or homologs thereof that maintain p65 protein activity (e.g. within at least 50%, 80%, 90%, 95%, 96%, 97%, 98%, 99% or 100% activity compared to p65 protein). In embodiments, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring p65 protein polypeptide. In embodiments, p65 protein is the protein as identified by the UniProt reference number Q04206, or a variant, homolog or functional fragment thereof. In embodiments, p65 includes the amino acid sequence of SEQ ID NO:10. In embodiments, p65 has the amino acid sequence of SEQ ID NO:10. In embodiments, p65 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:10. In embodiments, p65 has an amino acid sequence that has at least 85% sequence identity to SEQ ID NO:10. In embodiments, p65 has an amino acid sequence that has at least 90% sequence identity to SEQ ID NO:10. In embodiments, p65 has an amino acid sequence that has at least 92% sequence identity to SEQ ID NO:10. In embodiments, p65 has an amino acid sequence that has at least 94% sequence identity to SEQ ID NO:10. In embodiments, p65 has an amino acid sequence that has at least 95% sequence identity to SEQ ID NO:10. In embodiments, p65 has an amino acid sequence that has at least 96% sequence identity to SEQ ID NO:10. In embodiments, p65 has an amino acid sequence that has at least 98% sequence identity to SEQ ID NO:10.

The term “replication and transcription activator” or “Rta” or “Rta protein” or “R transactivator” as provided herein includes any of the recombinant or naturally-occurring forms Rta, or variants or homologs thereof that maintain Rta protein activity (e.g., within at least 50%, 80%, 90%, 95%, 96%, 97%, 98%, 99% or 100% activity compared to Rta protein). In embodiments, the variants or homologs have at least 90%, 95%, 96%, 97%, 98%, 99% or 100% amino acid sequence identity across the whole sequence or a portion of the sequence (e.g. a 50, 100, or 150 continuous amino acid portion) compared to a naturally occurring Rta protein polypeptide. In embodiments, Rta protein is the protein as identified by the UniProt reference number P03209, or a variant, homolog or functional fragment thereof. In embodiments, Rta includes the amino acid sequence of SEQ ID NO:11. In embodiments, Rta has the amino acid sequence of SEQ ID NO:11. In embodiments, Rta has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:11. In embodiments, Rta has an amino acid sequence that has at least 85% sequence identity to SEQ ID NO:11. In embodiments, Rta has an amino acid sequence that has at least 90% sequence identity to SEQ ID NO:11. In embodiments, Rta has an amino acid sequence that has at least 92% sequence identity to SEQ ID NO:11. In embodiments, Rta has an amino acid sequence that has at least 94% sequence identity to SEQ ID NO:11. In embodiments, Rta has an amino acid sequence that has at least 95% sequence identity to SEQ ID NO:11. In embodiments, Rta has an amino acid sequence that has at least 96% sequence identity to SEQ ID NO:11. In embodiments, Rta has an amino acid sequence that has at least 98% sequence identity to SEQ ID NO:11.

A “nuclear localization sequence” or “nuclear localization signal” or “NLS” is a peptide that directs proteins to the nucleus. In embodiments, the NLS includes five basic, positively charged amino acids. The NLS may be located anywhere on the peptide chain. In embodiments, the NLS is an NLS derived from SV40. In embodiments, the NLS is an NLS of nucleoplasmin. In embodiments, the NLS includes the sequence set forth by SEQ ID NO:12. In embodiments, the NLS is the sequence set forth by SEQ ID NO:12. In embodiments, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:12. In embodiments, NLS has an amino acid sequence that has at least 85% sequence identity to SEQ ID NO:12. In embodiments, NLS has an amino acid sequence that has at least 90% sequence identity to SEQ ID NO:12. In embodiments, NLS has an amino acid sequence that has at least 92% sequence identity to SEQ ID NO:12. In embodiments, NLS has an amino acid sequence that has at least 94% sequence identity to SEQ ID NO:12. In embodiments, NLS has an amino acid sequence that has at least 95% sequence identity to SEQ ID NO:12. In embodiments, NLS has an amino acid sequence that has at least 96% sequence identity to SEQ ID NO:12. In embodiments, NLS has an amino acid sequence that has at least 98% sequence identity to SEQ ID NO:12. In embodiments, the NLS includes the sequence set forth by SEQ ID NO:13. In embodiments, the NLS is the sequence set forth by SEQ ID NO:13. In embodiments, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:13. In embodiments, NLS has an amino acid sequence that has at least 85% sequence identity to SEQ ID NO:13. In embodiments, NLS has an amino acid sequence that has at least 90% sequence identity to SEQ ID NO:13. In embodiments, NLS has an amino acid sequence that has at least 92% sequence identity to SEQ ID NO:13. In embodiments, NLS has an amino acid sequence that has at least 94% sequence identity to SEQ ID NO:13. In embodiments, NLS has an amino acid sequence that has at least 95% sequence identity to SEQ ID NO:13. In embodiments, NLS has an amino acid sequence that has at least 96% sequence identity to SEQ ID NO:13. In embodiments, NLS has an amino acid sequence that has at least 98% sequence identity to SEQ ID NO:13.

“Epitope tag” refers to a biological moiety, such as a peptide, that is genetically engineered into a fusion protein and that functions as a universal epitope that is easily detected by commercially available assays or antibodies and that generally does not compromise the native structure or function of the protein. In embodiments, the fusion proteins described herein comprise one or more epitope tags.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide nanoparticles, USPIO nanoparticle aggregates, superparamagnetic iron oxide nanoparticles, SPIO nanoparticle aggregates, monochrystalline iron oxide nanoparticles, monochrystalline iron oxide, nanoparticle contrast agents, liposomes or other delivery vehicles containing gadolinium chelate molecules, gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gases, perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A detectable moiety is a monovalent detectable agent or a detectable agent capable of forming a bond with another composition. In embodiments, the detectable agent is an epitope tag. In embodiments, the epitope tag is an HA tag. In embodiments, the epitope tag is a FLAG-tag. In embodiments, the epitope tag comprises SEQ ID NO:17. In embodiments, the epitope tag is SEQ ID NO:17. In embodiments, the detectable agent is a fluorescent protein.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “connexin 43” or “Cx43” or “gap junction alpha-1 protein” refer to a protein that is encoded by the GJA1 gene on chromosome 6 which is involved with the regulation of cell death, proliferation, and differentiation. Connexin 43 includes any of the recombinant or naturally-occurring forms of connexin 43 or variants or homologs thereof that maintain connexin 43 activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to connexin 43). In embodiments, the variants or homologs have at least 50%, 55%, 60%, 65%, 70%, 75%, 80%, 85%, 90%, 91%, 92%, 93%, 94%, 95%, 96%, 97%, 98%, 99% or 100% amino acid sequence identity across the whole sequence or a portion of the sequence compared to a naturally occurring connexin 43 protein. In embodiments, the connexin 43 protein is SEQ ID NO:18 or a variant or homolog having substantial identity thereto. In embodiments, the connexin 43 protein is SEQ ID NO:18. In embodiments, the connexin 43 protein is SEQ ID NO:18. In embodiments, the connexin 43 protein has at least 90% sequence identity to SEQ ID NO:18. In embodiments, the connexin 43 protein has at least 92% sequence identity to SEQ ID NO:18. In embodiments, the connexin 43 protein has at least 94% sequence identity to SEQ ID NO:18. In embodiments, the connexin 43 protein has at least 95% sequence identity to SEQ ID NO:18. In embodiments, the connexin 43 protein has at least 96% sequence identity to SEQ ID NO:18. In embodiments, the connexin 43 protein has at least 98% sequence identity to SEQ ID NO:18. In embodiments, the connexin 43 protein has at least 90% sequence identity to SEQ ID NO:18, and has a serine to alanine mutation at the position corresponding to position 368 (i.e., an S368A mutation). In embodiments, the connexin 43 protein has at least 92% sequence identity to SEQ ID NO:18, and has an S368A mutation. In embodiments, the connexin 43 protein has at least 94% sequence identity to SEQ ID NO:18, and has an S368A mutation. In embodiments, the connexin 43 protein has at least 95% sequence identity to SEQ ID NO:18, and has an S368A mutation. In embodiments, the connexin 43 protein has at least 96% sequence identity to SEQ ID NO:18, and has an S368A mutation. In embodiments, the connexin 43 protein has at least 98% sequence identity to SEQ ID NO:18, and has an S368A mutation. In embodiments, the connexin 43 protein is SEQ ID NO:18.

In embodiments, the connexin 43 protein is SEQ ID NO:19 or a variant or homolog having substantial identity thereto. In embodiments, the connexin 43 protein is SEQ ID NO:19. In embodiments, the connexin 43 protein is SEQ ID NO:19. In embodiments, the connexin 43 protein has at least 90% sequence identity to SEQ ID NO:19. In embodiments, the connexin 43 protein has at least 92% sequence identity to SEQ ID NO:19. In embodiments, the connexin 43 protein has at least 94% sequence identity to SEQ ID NO:19. In embodiments, the connexin 43 protein has at least 95% sequence identity to SEQ ID NO:19. In embodiments, the connexin 43 protein has at least 96% sequence identity to SEQ ID NO:19. In embodiments, the connexin 43 protein has at least 98% sequence identity to SEQ ID NO:19. In embodiments, the connexin 43 protein is SEQ ID NO:19. In embodiments, SEQ ID NO:19 has a mutation at the position corresponding to position 368. In embodiments, SEQ ID NO:19 and has a serine to alanine mutation at the position corresponding to position 368, i.e. an S368A mutation.

As may be used herein, the terms “nucleic acid,” “nucleic acid molecule,” “nucleic acid oligomer,” “oligonucleotide,” “nucleic acid sequence,” “nucleic acid fragment,” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may have natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “amino acid sequence,” “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

A “fusion protein” refers to a chimeric protein encoding two or more separate protein sequences that are recombinantly expressed as a single moiety.

The terms “numbered with reference to” or “corresponding to” or “aligned with” when used in the context of the numbering of a given amino acid or polynucleotide sequence, refers to the numbering of the residues of a specified reference sequence when the given amino acid or polynucleotide sequence is compared to the reference sequence.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M).

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical,” “percent identity,” “sequence identity,” “homology,” or “sequence homology” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. In embodiments, identity exists over a region that is at least about 10, 15, 20, or 25 amino acids or nucleotides in length, or more preferably over a region that is 50-100 amino acids or nucleotides in length. Optionally, the identity exists over a region that is at least about 50 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

The terms “variant” or “derivative” in the context of polynucleotide (e.g. nucleic acid sequence or oligonucleotide) or peptide (e.g. an amino acid sequence or protein) refers to a polynucleotide sequence or peptide sequence that has some sequence similarity to their reference sequence. In embodiments, a variant or derivative can have at least 30%, 35%, 40%, 45%, 50%, 55%, 60%, 65%, 70%, 75%, 80%, 85%, 90%, 95%, 96%, 97%, 98% or 99% sequence identity (or equivalently used with similarity or homology) to its reference sequence. The terms “functional derivative” or “functional variant” in the context of polynucleotide or peptide sequence may refer to any variant or derivative that maintains the activity to a substantial level, e.g. at least 30% or more of the activity of the reference sequence.

A “vector” as used herein is a nucleic acid molecule that can be used as a vehicle to transfer genetic material into a cell. In embodiments, a vector refers to a DNA molecule harboring at least one origin of replication, a multiple cloning site (MCS) and one or more selection markers. A vector is typically composed of a backbone region and at least one insert or transgene region or a region designed for insertion of a DNA fragment or transgene such as a MCS. The backbone region often contains an origin of replication for propagation in at least one host and one or more selection markers. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment which do not require the use of recombination, transpositions or restriction enzymes (such as, but not limited to, uracil N glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. In embodiments, a vector contains additional features. Such additional features may include natural or synthetic promoters, genetic markers, antibiotic resistance cassettes or selection markers (e.g., toxins such as ccdB or tse2), epitopes or tags for detection, manipulation or purification (e.g., V5 epitope, c-myc, hemagglutinin (HA), FLAG™, polyhistidine (His), glutathione-S-transferase (GST), maltose binding protein (MBP)), scaffold attachment regions (SARs) or reporter genes (e.g., green fluorescent protein (GFP), red fluorescence protein (RFP), luciferase, β-galactosidase etc.). In embodiments, vectors are used to isolate, multiply or express inserted DNA fragments in a target host.

An “expression vector” is designed for expression of a transgene and generally harbors at least one promoter sequence that drives expression of the transgene. An expression vector is typically designed to contain one or more regulatory sequences such as enhancer, promoter and terminator regions that control expression of the inserted transgene. Suitable expression vectors include, without limitation, plasmids and viral vectors. In embodiments, the viral vector is a lentiviral vector. Vectors and expression systems for various applications are available from commercial suppliers such as Novagen (Madison, WI), Clontech (Palo Alto, CA), Stratagene (La Jolla, CA), and Life Technologies Corp. (Carlsbad, CA).

A “promoter” as used herein is a transcription regulatory sequence which is capable of directing transcription of a nucleic acid segment (e.g., a transgene having, for example, an open reading frame) when operably connected thereto. The choice of a promoter to be included in an vector depends upon several factors, including without limitation efficiency, selectability, inducibility, desired expression level, and cell or tissue specificity. For example, tissue-, organ- and cell-specific promoters that confer transcription only or predominantly in a particular tissue, organ, and cell type, respectively, can be used. Other classes of promoters include, but are not limited to, inducible promoters, such as promoters that confer transcription in response to external stimuli such as chemical agents, developmental stimuli, or environmental stimuli. Inducible promoters may be induced by pathogens or stress like cold, heat, UV light, or high ionic concentrations or may be induced by chemicals. Examples of inducible promoters are the eukaryotic metallothionein promoter, which is induced by increased levels of heavy metals; the prokaryotic lacL promoter, which is induced in response to isopropyl-β-D-thiogalacto-pyranoside; and eukaryotic heat shock promoters, which are induced by raised temperature. Numerous additional bacterial and eukaryotic promoters suitable for use with the invention are known in the art and described in, e.g., in Sambrook et al., Molecular Cloning, A Laboratory Manual (2nd ed. 1989; 3rd ed., 2001); Kriegler, Gene Transfer and Expression: A Laboratory Manual (1990); and Ausubel et al., Current Protocols in Molecular Biology. Bacterial expression systems for expressing the fusion proteins described herein are available in, e.g., E. coli, Bacillus sp., and Salmonella (Palva et al. Secretion of interferon by Bacillus subtilis. Gene 22:229-235 (1983)). Kits for such expression systems are commercially available. Eukaryotic expression systems for mammalian cells, yeast, and insect cells are well known by those of skill in the art and are also commercially available.

Common promoters for prokaryotic protein expression are e.g., lac promoter or trc and tac promoter (IPTG induction), tetA promoter/operator (anhydrotetracyclin induction), PPBAD promoter (L-arabinose induction), r/zaPBAD promoter (L-rhamnose induction) or phage promoters such as phage promoter pL (temperature shift sensitive), T7, T3, SP6, or T5.

Common promoters for mammalian protein expression are, e.g., cytomegalovirus (CMV) promoter, H1 promoter, EF1 alpha promoter, SV40 promoter/enhancer, Vaccinia virus promoter, Viral LTRs (MMTV, RSV, HIV etc.), EIB promoter, promoters of constitutively expressed genes (actin, GAPDH), promoters of genes expressed in a tissue-specific manner (albumin, NSE), promoters of inducible genes (Metallothionein, steroid hormones).

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “purified” denotes that a nucleic acid or protein gives rise to essentially one band in an electrophoretic gel. In embodiments, the nucleic acid or protein is at least 50% pure, at least 65% pure, at least 75% pure, at least 85% pure, at least 95% pure, or at least 99% pure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. In embodiments, the cell is a human cell.

The term “allogenic cells” refers to cells that are taken from a donor, manipulated (e.g., contacted with a nucleic acid as described herein), and then administered to a patient who is different from the donor to treat a disease (e.g., cystic fibrosis).

The term “autologous cells” refers to cells that are taken from a patient, manipulated (e.g., contacted with a nucleic acid as described herein), and then administered to the same patient to treat a disease (e.g., cystic fibrosis).

The term “culture” or “cell culture” means the maintenance of cells in an artificial, in vitro environment. A “cell culture system” is used herein to refers to culture conditions in which a population of cells are contacted with nucleic acids under conditions which allow for the exogenous polypeptide to enter into the cells, e.g., via endocytosis. “Culture medium” is used herein to refer to a nutrient solution for the culturing, growth, or proliferation of cells.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection,” “transduction,” “transfecting,” or “transducing” can be used interchangeably and are defined as a process of introducing a nucleic acid molecule and/or a protein to a cell. Nucleic acids may be introduced to a cell using various methods. The nucleic acid molecule can be a sequence encoding complete proteins or functional portions thereof. Typically, a vector, comprising the elements necessary for protein expression (e.g., a promoter, transcription start site, etc.). Exemplary transfection methods include exosome transfection, calcium phosphate transfection, liposomal transfection, nucleofection, sonoporation, transfection through heat shock, magnetifection and electroporation. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “specific binding,” “specifically bind,” or “specifically binds” refer to two molecules (e.g., DNA-binding domain and its specific binding (or targeting) nucleic acid sequence) that bind to each other with a higher affinity and specificity than a binding between random (e.g. non-target) molecules.

As used herein, the phrase “recognition sequence,” “recognition site,” “target sequence” or “target site” refers to a particular sequence to which a protein, chemical compound, DNA, or RNA molecule (e.g., a DNA binding domain such as zinc finger domain) recognizes and binds. A recognition sequence or target sequence may refer to a nucleic acid sequence, DNA or RNA, that is recognized and bound by a fusion protein with specificity. In certain examples, the recognition sequence or target sequence may be a nucleic acid sequence from the CFTR gene that is either integrated into the host cell's genome or present as a separate nucleic acid molecule (i.e., episome). Therefore, in some examples where the fusion protein has transcription activity, the transcription of the recognition sequence (or the target sequence) or a sequence having the recognition sequence (or the target sequence) can be activated by the fusion protein.

As defined herein, the term “activation,” “activate,” “activating” and the like in reference to gene expression or transcription refers to conversion of a gene or nucleic acid sequence to be transcribed to its complementary RNA (e.g. mRNA) from an initially inactive or deactivated state. In embodiments, the gene or nucleic acid sequence that is normally transcribed to a certain extent is activated such that its transcription level is enhanced or increased as compared to its normal level of transcription.

The term “activation,” “activate,” “activating,” “reactivation,” “reactivate,” “reactivating” and the like used in the context of virus infection refers to enhancing, promoting, stimulating or increasing the activity of the virus including the transcriptional activation of viral gene(s) or genome. Especially in the context of activation or reactivation of latent virus, the term may refer to (i) initiation of transcription of certain viral genes that were previously transcriptionally inactive, (ii) increase of existing transcription of viral genes and/or (iii) transcription of a viral genome. With this activation or reactivation, the latent virus may become transcriptionally active, replicating the viral genome and producing viral progenies such that the virus is no longer in the latency and enters into the lytic cycle.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this document, unless the context requires otherwise, the words “comprise,” “comprising,” “contain,” “containing,” “have,” “having,” “include,” or “including” will be understood to imply the inclusion of a stated step or element or group of steps or elements but not the exclusion of any other step or element or group of steps or elements.

Cystic fibrosis is a genetic disorder that results in a multi-organ disease with progressive respiratory decline which leads to premature death. Mutations in the cystic fibrosis transmembrane conductance regulator (CFTR) gene disrupts the capacity of the protein to function as a channel, transporting chloride ions and bicarbonate across epithelial cell membranes. Small molecule treatments targeted at potentiating or correcting CFTR have shown clinical benefits, but are only effective for a small percentage of individuals with specific CFTR mutations. To overcome this limitation, we engineered stromal-derived mesenchymal stem cells (MSC) to produce exosomes containing a novel CFTR Zinc Finger Protein fusion with transcriptional activation domains VP64, p65 and Rta to target the CFTR promoter (CFZF-VPR) and activate transcription. Treatment with CFZF-VPR results in robust activation of CFTR transcription in patient derived Human Bronchial Epithelial cells (HuBEC). We also find that CFZF-VPR can be packaged and delivered to HuBEC and HEK293 cell lines by MSC derived exosomes to potently activate CFTR expression. Connexin 43 appeared to be required for functional release of CFZF-VPR from exosomes. The observations presented here demonstrate that MSC derived exosomes can be used to deliver a packaged zinc finger activator to target cells and activate CFTR. The novel approach presented here offers a next-generation genetic therapy that may one day prove effective in treating patients afflicted with Cystic fibrosis.

Exosomes are important mediators that facilitate intercellular communication and transfer of genetic material to recipient cells. Here, we take advantage of this inherent property and engineer MSCs to produce exosomes packaged with a Zinc Finger Activator targeted to the CFTR gene promoter. We find that treatment of CF patient derived HuBEC with the CFZF-VPR containing exosomes results in significant activation of CFTR. The novel approach presented here represents an effective way to deliver therapeutic proteins to patients afflicted with CF.

Zinc Finger Domain

The disclosure provides a zinc finger domain having at least 85% sequence identity to SEQ ID NO:8. In embodiments, the zinc finger domain has an amino acid sequence that has at least 90% sequence identity to SEQ ID NO:8. In embodiments, the zinc finger domain has an amino acid sequence that has at least 92% sequence identity to SEQ ID NO:8. In embodiments, the zinc finger domain has an amino acid sequence that has at least 94% sequence identity to SEQ ID NO:8. In embodiments, the zinc finger domain has an amino acid sequence that has at least 95% sequence identity to SEQ ID NO:8. In embodiments, the zinc finger domain has an amino acid sequence that has at least 96% sequence identity to SEQ ID NO:8. In embodiments, the zinc finger domain has an amino acid sequence that has at least 98% sequence identity to SEQ ID NO:8. In embodiments, the zinc finger domain is capable of binding to the a CFTR gene. In embodiments, the zinc finger domain is capable of binding to the promoter sequence of a CFTR gene. In embodiments, the zinc finger domain is capable of binding to the promoter sequence of a CFTR gene, wherein the promoter sequence has at least 90% sequence identity to SEQ ID NO:1. In embodiments, the zinc finger domain is capable of binding to the promoter sequence of a CFTR gene, wherein the promoter sequence has at least 95% sequence identity to SEQ ID NO:1. In embodiments, the zinc finger domain is capable of binding to the promoter sequence of a CFTR gene, wherein the promoter sequence has SEQ ID NO:1.

Fusion Proteins

The disclosure provides a fusion protein comprises a zinc finger domain and a transcriptional activator recognizes (or binds to) a target nucleic acid sequence with specificity. The target nucleic acid can be DNA or RNA sequence. In embodiments, the target nucleic acid sequence is a CFTR gene. In embodiments, the target nucleic acid sequence is the promoter sequence of the CFTR gene. In embodiments, the fusion protein comprises a zinc finger domain and a transcriptional activator, wherein the fusion protein is capable of binding to a cystic fibrosis transmembrane conductance regulator (CFTR) gene. In embodiments, the fusion protein comprises a zinc finger domain and a transcriptional activator, wherein the fusion protein is capable of binding to the promoter sequence of the CFTR gene. In embodiments, the CFTR gene has a deletion of a phenylalanine residue at position 508 (F508del). In embodiments, the promoter sequence of the CFTR gene comprises SEQ ID NO. 1. In embodiments, the promoter sequence of the CFTR gene has at least 50%, 55%, 60%, 65%, 70%, 75%, 80%, 85%, 90%, 95%, 96%, 97%, 98% or 99% sequence identity to the sequence of SEQ ID NO.1. In embodiments, the promoter sequence of the CFTR gene has at least 90% sequence identity to SEQ ID NO:1. In embodiments, the promoter sequence of the CFTR gene has at least 92% sequence identity to SEQ ID NO:1. In embodiments, the promoter sequence of the CFTR gene has at least 94% sequence identity to SEQ ID NO:1. In embodiments, the promoter sequence of the CFTR gene has at least 95% sequence identity to SEQ ID NO:1. In embodiments, the promoter sequence of the CFTR gene has at least 96% sequence identity to SEQ ID NO:1. In embodiments, the promoter sequence of the CFTR gene has at least 97% sequence identity to SEQ ID NO:1. In embodiments, the promoter sequence of the CFTR gene has at least 98% sequence identity to SEQ ID NO:1. In embodiments, the promoter sequence of the CFTR gene has at least 99% sequence identity to SEQ ID NO:1. In embodiments, the promoter sequence of the CFTR gene is SEQ ID NO. 1.

The disclosure provides a fusion protein comprising a zinc finger domain; wherein the zinc finger domain has at least 90% sequence identity to SEQ ID NO:8; and wherein the fusion protein is capable of binding to the promoter sequence of a CFTR gene. In embodiments, the zinc finger domain has at least 90% sequence identity to SEQ ID NO:8. In embodiments, the zinc finger domain has at least 92% sequence identity to SEQ ID NO:8. In embodiments, the zinc finger domain has at least 94% sequence identity to SEQ ID NO:8. In embodiments, the zinc finger domain has at least 95% sequence identity to SEQ ID NO:8. In embodiments, the zinc finger domain has at least 96% sequence identity to SEQ ID NO:8. In embodiments, the zinc finger domain has at least 98% sequence identity to SEQ ID NO:8. In embodiments, the zinc finger domain has SEQ ID NO:8. In embodiments, the promoter sequence of the CFTR gene has at least 95% sequence identity to SEQ ID NO:1. In embodiments, the promoter sequence of the CFTR gene has at least 96% sequence identity to SEQ ID NO:1. In embodiments, the promoter sequence of the CFTR gene has at least 98% sequence identity to SEQ ID NO:1. In embodiments, the promoter sequence of the CFTR gene has SEQ ID NO:1. In embodiments, the fusion protein further comprises a transcriptional activator. In embodiments, the transcriptional activator comprises VP64, p65, Rta, or a combination of two or more thereof. In embodiments, the transcriptional activator comprises VP64, p65, and Rta.

In embodiments, the fusion protein comprises, from N-terminus to C-terminus, a zinc finger domain having SEQ ID NO:8, VP64 having SEQ ID NO:9, p65 having SEQ ID NO:10, and Rta having SEQ ID NO:11. In embodiments, the fusion protein further comprises one or more peptide linkers. In embodiments, the fusion protein further comprises one or more glycine-serine linkers. In embodiments, the fusion protein further comprises a nuclear localization signal (e.g., SEQ ID NO:12 or SEQ ID NO:13), a TAT domain (e.g., of SEQ ID NO:14 or SEQ ID NO:15); an Myc tag (e.g., of SEQ ID NO:16), or a combination of two or more thereof.

In embodiments, the fusion protein has an amino acid sequence with at least 85% identity to the amino acid sequence of a fusion protein comprising, from N-terminus to C-terminus, a zinc finger domain having SEQ ID NO:8, VP64 having SEQ ID NO:9, p65 having SEQ ID NO:10, and Rta having SEQ ID NO:11. In embodiments, the fusion protein further comprises one or more peptide linkers. In embodiments, the fusion protein further comprises one or more glycine-serine linkers. In embodiments, the fusion protein further comprises a nuclear localization signal (e.g., SEQ ID NO:12 or SEQ ID NO:13), a TAT domain (e.g., of SEQ ID NO:14 or SEQ ID NO:15); an Myc tag (e.g., of SEQ ID NO:16), or a combination of two or more thereof. In embodiments, the fusion protein has at least 90% sequence identity to the amino acid sequence of the fusion protein described herein. In embodiments, the fusion protein has at least 92% sequence identity to the amino acid sequence of the fusion protein described herein. In embodiments, the fusion protein has at least 94% sequence identity to the amino acid sequence of the fusion protein described herein. In embodiments, the fusion protein has at least 95% sequence identity to the amino acid sequence of the fusion protein described herein. In embodiments, the fusion protein has at least 96% sequence identity to the amino acid sequence of the fusion protein described herein. In embodiments, the fusion protein has at least 98% sequence identity to the amino acid sequence of the fusion protein described herein.

In embodiments, the fusion protein has an amino acid sequence comprising, from N-terminus to C-terminus, a nuclear localization signal having SEQ ID NO:12 or SEQ ID NO:13, a zinc finger domain having SEQ ID NO:8, VP64 having SEQ ID NO:9, p65 having SEQ ID NO:10, and Rta having SEQ ID NO:11. In embodiments, the nuclear localization signal has SEQ ID NO:12. In embodiments, the nuclear localization signal has SEQ ID NO:13. In embodiments, the fusion protein further comprises one or more peptide linkers. In embodiments, the fusion protein further comprises one or more glycine-serine linkers. In embodiments, the fusion protein further comprises a TAT domain (e.g., of SEQ ID NO:14 or SEQ ID NO:15); an Myc tag (e.g., of SEQ ID NO:16), or a combination thereof.

In embodiments, the fusion protein has an amino acid sequence with at least 85% identity to the amino acid sequence of a fusion protein comprising, from N-terminus to C-terminus, a nuclear localization signal having SEQ ID NO:12 or SEQ ID NO:13, a zinc finger domain having SEQ ID NO:8, VP64 having SEQ ID NO:9, p65 having SEQ ID NO:10, and Rta having SEQ ID NO:11. In embodiments, the nuclear localization signal has SEQ ID NO:12. In embodiments, the nuclear localization signal has SEQ ID NO:13. In embodiments, the fusion protein further comprises one or more peptide linkers. In embodiments, the fusion protein further comprises one or more glycine-serine linkers. In embodiments, the fusion protein further comprises a TAT domain (e.g., of SEQ ID NO:14 or SEQ ID NO:15); an Myc tag (e.g., of SEQ ID NO:16), or a combination thereof. In embodiments, the fusion protein has at least 90% sequence identity to the amino acid sequence of the fusion protein described herein. In embodiments, the fusion protein has at least 92% sequence identity to the amino acid sequence of the fusion protein described herein. In embodiments, the fusion protein has at least 94% sequence identity to the amino acid sequence of the fusion protein described herein. In embodiments, the fusion protein has at least 95% sequence identity to the amino acid sequence of the fusion protein described herein. In embodiments, the fusion protein has at least 96% sequence identity to the amino acid sequence of the fusion protein described herein. In embodiments, the fusion protein has at least 98% sequence identity to the amino acid sequence of the fusion protein described herein.

In embodiments, the fusion protein has an amino acid sequence comprising, from N-terminus to C-terminus, a first nuclear localization signal having SEQ ID NO:12 or SEQ ID NO:13, a zinc finger domain having SEQ ID NO:8, VP64 having SEQ ID NO:9, a second nuclear localization signal having SEQ ID NO:12 or SEQ ID NO:13, p65 having SEQ ID NO:10, and Rta having SEQ ID NO:11. In embodiments, the nuclear localization signal has SEQ ID NO:12. In embodiments, the nuclear localization signal has SEQ ID NO:13. In embodiments, the first and/or second nuclear localization signal has SEQ ID NO:12. In embodiments, the first and/or second nuclear localization signal has SEQ ID NO:13. In embodiments, the first nuclear localization signal has SEQ ID NO:12 and the second nuclear localization signal has SEQ ID NO:13. In embodiments, the fusion protein further comprises one or more peptide linkers. In embodiments, the fusion protein further comprises one or more glycine-serine linkers. In embodiments, the fusion protein further comprises a TAT domain (e.g., of SEQ ID NO:14 or SEQ ID NO:15); an Myc tag (e.g., of SEQ ID NO:16), or a combination thereof.

In embodiments, the fusion protein has an amino acid sequence with at least 85% identity to the amino acid sequence of a fusion protein comprising, from N-terminus to C-terminus, a first nuclear localization signal having SEQ ID NO:12 or SEQ ID NO:13, a zinc finger domain having SEQ ID NO:8, VP64 having SEQ ID NO:9, a second nuclear localization signal having SEQ ID NO:12 or SEQ ID NO:13, p65 having SEQ ID NO:10, and Rta having SEQ ID NO:11. In embodiments, the first and/or second nuclear localization signal has SEQ ID NO:12. In embodiments, the first and/or second nuclear localization signal has SEQ ID NO:13. In embodiments, the first nuclear localization signal has SEQ ID NO:12 and the second nuclear localization signal has SEQ ID NO:13. In embodiments, the fusion protein further comprises one or more peptide linkers. In embodiments, the fusion protein further comprises one or more glycine-serine linkers. In embodiments, the fusion protein further comprises a TAT domain (e.g., of SEQ ID NO:14 or SEQ ID NO:15); an Myc tag (e.g., of SEQ ID NO:16), or a combination thereof. In embodiments, the fusion protein has at least 90% sequence identity to the amino acid sequence of the fusion protein described herein. In embodiments, the fusion protein has at least 92% sequence identity to the amino acid sequence of the fusion protein described herein. In embodiments, the fusion protein has at least 94% sequence identity to the amino acid sequence of the fusion protein described herein. In embodiments, the fusion protein has at least 95% sequence identity to the amino acid sequence of the fusion protein described herein. In embodiments, the fusion protein has at least 96% sequence identity to the amino acid sequence of the fusion protein described herein. In embodiments, the fusion protein has at least 98% sequence identity to the amino acid sequence of the fusion protein described herein.

In embodiments, the fusion protein has an amino acid sequence comprising, from N-terminus to C-terminus, a first nuclear localization signal having SEQ ID NO:12 or SEQ ID NO:13, a first peptide linker, a zinc finger domain having SEQ ID NO:8, VP64 having SEQ ID NO:9, a second nuclear localization signal having SEQ ID NO:12 or SEQ ID NO:13, a second peptide linker, p65 having SEQ ID NO:10, a third peptide linker, and Rta having SEQ ID NO:11. In embodiments, the nuclear localization signal has SEQ ID NO:12. In embodiments, the nuclear localization signal has SEQ ID NO:13. In embodiments, the first and/or second nuclear localization signal has SEQ ID NO:12. In embodiments, the first and/or second nuclear localization signal has SEQ ID NO:13. In embodiments, the first nuclear localization signal has SEQ ID NO:12 and the second nuclear localization signal has SEQ ID NO:13. In embodiments, the first, second, and third peptide linkers are each independently a glycine-serine linker. In embodiments, the fusion protein further comprises a TAT domain (e.g., of SEQ ID NO:14 or SEQ ID NO:15); an Myc tag (e.g., of SEQ ID NO:16), or a combination thereof.

In embodiments, the fusion protein has an amino acid sequence with at least 85% identity to the amino acid sequence of a fusion protein comprising, from N-terminus to C-terminus, a first nuclear localization signal having SEQ ID NO:12 or SEQ ID NO:13, a first peptide linker, a zinc finger domain having SEQ ID NO:8, VP64 having SEQ ID NO:9, a second nuclear localization signal having SEQ ID NO:12 or SEQ ID NO:13, a second peptide linker, p65 having SEQ ID NO:10, a third peptide linker, and Rta having SEQ ID NO:11. In embodiments, the first and/or second nuclear localization signal has SEQ ID NO:12. In embodiments, the first and/or second nuclear localization signal has SEQ ID NO:13. In embodiments, the first nuclear localization signal has SEQ ID NO:12 and the second nuclear localization signal has SEQ ID NO:13. In embodiments, the first, second, and third peptide linkers are each independently a glycine-serine linker. In embodiments, the fusion protein further comprises a TAT domain (e.g., of SEQ ID NO:14 or SEQ ID NO:15); an Myc tag (e.g., of SEQ ID NO:16), or a combination thereof. In embodiments, the fusion protein has at least 90% sequence identity to the amino acid sequence of the fusion protein described herein. In embodiments, the fusion protein has at least 92% sequence identity to the amino acid sequence of the fusion protein described herein. In embodiments, the fusion protein has at least 94% sequence identity to the amino acid sequence of the fusion protein described herein. In embodiments, the fusion protein has at least 95% sequence identity to the amino acid sequence of the fusion protein described herein. In embodiments, the fusion protein has at least 96% sequence identity to the amino acid sequence of the fusion protein described herein. In embodiments, the fusion protein has at least 98% sequence identity to the amino acid sequence of the fusion protein described herein.

A “peptide linker” as provided herein is a linker including a peptide moiety. In embodiments, the peptide linker is a divalent peptide, such as an amino acid sequence attached at the N-terminus and the C-terminus to the remainder of the compound (e.g., fusion protein provided herein). In embodiments, the peptide linker is a glycine-serine linker. In embodiments, the peptide linker includes 1 to about 30 amino acid residues. In embodiments, the peptide linker includes 1 to about 25 amino acid residues. In embodiments, the peptide linker includes 1 to about 20 amino acid residues. In embodiments, the peptide linker includes about 2 to about 20 amino acid residues. In embodiments, the peptide linker includes about 2 to about 19 amino acid residues. In embodiments, the peptide linker includes about 2 to about 18 amino acid residues. In embodiments, the peptide linker includes about 2 to about 17 amino acid residues. In embodiments, the peptide linker includes about 2 to about 16 amino acid residues. In embodiments, the peptide linker includes about 2 to about 15 amino acid residues. In embodiments, the peptide linker includes about 2 to about 14 amino acid residues. In embodiments, the peptide linker includes about 2 to about 13 amino acid residues. In embodiments, the peptide linker includes about 2 to about 12 amino acid residues. In embodiments, the peptide linker includes about 2 to about 11 amino acid residues. In embodiments, the peptide linker includes about 2 to about 10 amino acid residues. In embodiments, the peptide linker includes about 2 to about 9 amino acid residues. In embodiments, the peptide linker includes about 2 to about 8 amino acid residues. In embodiments, the peptide linker includes about 2 to about 7 amino acid residues. In embodiments, the peptide linker includes about 2 to about 6 amino acid residues. In embodiments, the peptide linker includes about 2 to about 5 amino acid residues. In embodiments, the peptide linker includes about 2 to about 4 amino acid residues. In embodiments, the peptide linker includes about 2 to about 3 amino acid residues.

A “glycine-serine linker” refers to a peptide linker comprising glycine and serine. In embodiments, the glycine-serine linker comprises -(GS)ₓ-, where x is an integer from 1 to 20. In embodiments, x is an integer from 1 to 12. In embodiments, x is an integer from 1 to 10. In embodiments, x is an integer from 1 to 8. In embodiments, x is an integer from 1 to 6. In embodiments, x is an integer from 1 to 4. In embodiments, the glycine-serine linker is -GS-. In embodiments, the glycine-serine linker is -GSGS-. In embodiments, the glycine-serine linker is -GSGSGS-. In embodiments, the glycine-serine linker is -GSGSGSGS-. In embodiments, the glycine-serine linker is -GSGSGSGSGS-. In embodiments, the glycine-serine linker is -GSGSGSGSGSGS-. In embodiments, the glycine-serine linker comprises -SS(GS)ₓ-, where x is an integer from 1 to 20. In embodiments, the glycine-serine linker comprises -S(GS)ₓ-, where x is an integer from 1 to 20. In embodiments, x is an integer from 1 to 12. In embodiments, x is an integer from 1 to 10. In embodiments, x is an integer from 1 to 8. In embodiments, x is an integer from 1 to 6. In embodiments, x is an integer from 1 to 4. In embodiments, the glycine-serine linker is -SSGS-. In embodiments, the glycine-serine linker is -SG-. In embodiments, the glycine-serine linker is -SSG-.

In embodiments, the glycine-serine linker is -(G₄S)ₓ-, where x is an integer from 1 to 20. In embodiments, the glycine-serine linker is -(G₃S)ₓ-, where x is an integer from 1 to 20. In embodiments, the glycine-serine linker is -(G₂S)ₓ-, where x is an integer from 1 to 20. In embodiments, the glycine-serine linker is -S(G₄S)ₓ-, where x is an integer from 1 to 20. In embodiments, the glycine-serine linker is -S(G₃S)ₓ-, where x is an integer from 1 to 20. In embodiments, the glycine-serine linker is -S(G₂S)ₓ-, where x is an integer from 1 to 20. In embodiments of the glycine-serine linkers described herein, x is an integer from 1 to 12. In embodiments of the glycine-serine linkers described herein, x is an integer from 1 to 10. In embodiments of the glycine-serine linkers described herein, x is an integer from 1 to 8. In embodiments of the glycine-serine linkers described herein, x is an integer from 1 to 6. In embodiments of the glycine-serine linkers described herein, x is an integer from 1 to 4.

The disclosure provides a fusion protein comprising SEQ ID NO:2. In embodiments, the fusion protein has at least 85% sequence identity to SEQ ID NO:2. In embodiments, the fusion protein has at least 88% sequence identity to SEQ ID NO:2. In embodiments, the fusion protein has at least 90% sequence identity to SEQ ID NO:2. In embodiments, the fusion protein has at least 92% sequence identity to SEQ ID NO:2. In embodiments, the fusion protein has at least 94% sequence identity to SEQ ID NO:2. In embodiments, the fusion protein has at least 95% sequence identity to SEQ ID NO:2. In embodiments, the fusion protein has at least %% sequence identity to SEQ ID NO:2. In embodiments, the fusion protein has at least 98% sequence identity to SEQ ID NO:2. In embodiments, the fusion protein is SEQ ID NO:2.

The disclosure provides a fusion protein comprising SEQ ID NO:3. In embodiments, the fusion protein has at least 85% sequence identity to SEQ ID NO:3. In embodiments, the fusion protein has at least 88% sequence identity to SEQ ID NO:3. In embodiments, the fusion protein has at least 90% sequence identity to SEQ ID NO:3. In embodiments, the fusion protein has at least 92% sequence identity to SEQ ID NO:3. In embodiments, the fusion protein has at least 94% sequence identity to SEQ ID NO:3. In embodiments, the fusion protein has at least 95% sequence identity to SEQ ID NO:3. In embodiments, the fusion protein has at least 96% sequence identity to SEQ ID NO:3. In embodiments, the fusion protein has at least 98% sequence identity to SEQ ID NO:3. In embodiments, the fusion protein is SEQ ID NO:3.

The disclosure provides a fusion protein comprising SEQ ID NO:4. In embodiments, the fusion protein has at least 85% sequence identity to SEQ ID NO:4. In embodiments, the fusion protein has at least 88% sequence identity to SEQ ID NO:4. In embodiments, the fusion protein has at least 90% sequence identity to SEQ ID NO:4. In embodiments, the fusion protein has at least 92% sequence identity to SEQ ID NO:4. In embodiments, the fusion protein has at least 94% sequence identity to SEQ ID NO:4. In embodiments, the fusion protein has at least 95% sequence identity to SEQ ID NO:4. In embodiments, the fusion protein has at least 96% sequence identity to SEQ ID NO:4. In embodiments, the fusion protein has at least 98% sequence identity to SEQ ID NO:4. In embodiments, the fusion protein is SEQ ID NO:4.

The disclosure provides a fusion protein comprising SEQ ID NO:5. In embodiments, the fusion protein has at least 85% sequence identity to SEQ ID NO:5. In embodiments, the fusion protein has at least 88% sequence identity to SEQ ID NO:5. In embodiments, the fusion protein has at least 90% sequence identity to SEQ ID NO:5. In embodiments, the fusion protein has at least 92% sequence identity to SEQ ID NO:5. In embodiments, the fusion protein has at least 94% sequence identity to SEQ ID NO:5. In embodiments, the fusion protein has at least 95% sequence identity to SEQ ID NO:5. In embodiments, the fusion protein has at least 96% sequence identity to SEQ ID NO:5. In embodiments, the fusion protein has at least 98% sequence identity to SEQ ID NO:5. In embodiments, the fusion protein is SEQ ID NO:5.

The disclosure provides a fusion protein comprising SEQ ID NO:6. In embodiments, the fusion protein has at least 85% sequence identity to SEQ ID NO:6. In embodiments, the fusion protein has at least 88% sequence identity to SEQ ID NO:6. In embodiments, the fusion protein has at least 90% sequence identity to SEQ ID NO:6. In embodiments, the fusion protein has at least 92% sequence identity to SEQ ID NO:6. In embodiments, the fusion protein has at least 94% sequence identity to SEQ ID NO:6. In embodiments, the fusion protein has at least 95% sequence identity to SEQ ID NO:6. In embodiments, the fusion protein has at least 96% sequence identity to SEQ ID NO:6. In embodiments, the fusion protein has at least 98% sequence identity to SEQ ID NO:6. In embodiments, the fusion protein is SEQ ID NO:6.

The disclosure provides a fusion protein comprising SEQ ID NO:7. In embodiments, the fusion protein has at least 85% sequence identity to SEQ ID NO:7. In embodiments, the fusion protein has at least 88% sequence identity to SEQ ID NO:7. In embodiments, the fusion protein has at least 90% sequence identity to SEQ ID NO:7. In embodiments, the fusion protein has at least 92% sequence identity to SEQ ID NO:7. In embodiments, the fusion protein has at least 94% sequence identity to SEQ ID NO:7. In embodiments, the fusion protein has at least 95% sequence identity to SEQ ID NO:7. In embodiments, the fusion protein has at least 96% sequence identity to SEQ ID NO:7. In embodiments, the fusion protein has at least 98% sequence identity to SEQ ID NO:7. In embodiments, the fusion protein is SEQ ID NO:7.

In embodiments, the fusion protein of the present disclosure, including embodiments thereof, has further components, which include, but are not limited to, epitope tags, nuclear localization signals, cell-penetrating peptides (e.g., a TAT peptide or a derivative thereof), and the like. Additionally, a peptide that can promote stabilization of the fusion protein and/or enhance the protein isolation (e.g., myc-tag sequence and a maltose binding sequence) can also be contained in the fusion protein.

Cell-penetrating peptides (CPPs) generally are short peptides that can facilitate cellular intake/uptake of various molecular equipment (e.g. a fusion protein). The cargo is associated with the CPPs either through chemical linkage via covalent bonds or through non-covalent interactions. The function of the CPPs is to deliver the cargo into cells. Any peptides that are known to be capable of CPPs or have cell-penetrating activity can be used in the fusion proteins described herein. In embodiments, the trans-activating transcriptional activator (TAT) is used as a CPP, thereby enhancing the intake/uptake of the fusion protein into the cells. In embodiments, the CCP (TAT) is SEQ ID NO:14 or SEQ ID NO:15. In embodiments, the CCP (TAT) is SEQ ID NO:14. In embodiments, the CCP (TAT) is SEQ ID NO:15.

In embodiments, the fusion protein has one or more additional sequences such as a myc-tag sequence and/or maltose-binding sequence. A myc tag is a polypeptide tag derived from the c-myc gene product that can be added to a protein using recombinant DNA technology. It can be used for affinity chromatography, then used to separate fusion protein expressed by the host organism. It can also be used in the isolation of protein complexes with multiple subunits. In embodiments, the myc-tage is SEQ ID NO:16. Maltose binding peptide (MBP), which was originally found as an Escherichia coli gene and product thereof, can be used to increase the solubility of fusion protein. In this system, the fusion protein can be expressed as a MBP-fusion protein, preventing aggregation of the fusion protein. In addition, MBP can also be used as an affinity tag for purification of fusion proteins. Thus, the MBP-protein fusion can be purified by eluting the column with maltose. Once the fusion protein is obtained in purified form, the protein of interest can often be cleaved from MBP with a specific protease. The fusion protein can then be separated from MBP by affinity chromatography. In embodiments, the fusion protein has a MBP sequence that is known in the art or a derivative thereof.

Complexes

The disclosure provides a complex comprising a fusion protein described herein, including all embodiments thereof, and a CFTR gene. In embodiments, the fusion protein is bonded to the CFTR gene. In embodiments, the fusion protein is non-covalently bonded to the CFTR gene. In embodiments, the fusion protein is ionically bonded to the CFTR gene. In embodiments, the complex comprises a fusion protein described herein, including all embodiments thereof, bonded to the promoter sequence of a CFTR gene. In embodiments, the fusion protein is non-covalently bonded to promoter sequence of the CFTR gene. In embodiments, the fusion protein is ionically bonded to promoter sequence of the CFTR gene. In embodiments, the CFTR gene has a deletion of a phenylalanine residue at position 508 (F508del).

Nucleic Acids

The disclosure provides nucleic acids encoding the fusion proteins described herein, including embodiments thereof. In embodiments, the nucleic acid sequence encodes for a fusion protein described herein, including fusion proteins having amino acid sequences with certain percent sequence identities described herein, including embodiments thereof. In embodiments, the nucleic acid is RNA. In embodiments, the nucleic acid is messenger RNA. In embodiments, the nucleic acid is DNA. In embodiments, the disclosure provides a nucleic acid sequence that encodes a fusion protein comprising a zinc finger domain and a transcriptional activator, wherein the fusion protein is capable of binding to the promoter sequence of the cystic fibrosis transmembrane conductance regulator gene.

In embodiments, the disclosure provides a nucleic acid sequence that encodes the fusion protein of SEQ ID NO:2, including all embodiments and percent sequence identities described herein. In embodiments, the disclosure provides a nucleic acid sequence that encodes the fusion protein of SEQ ID NO:3, including all embodiments and percent sequence identities described herein. In embodiments, the disclosure provides a nucleic acid sequence that encodes the fusion protein of SEQ ID NO:4, including all embodiments and percent sequence identities described herein. In embodiments, the disclosure provides a nucleic acid sequence that encodes the fusion protein of SEQ ID NO:5, including all embodiments and percent sequence identities described herein. In embodiments, the disclosure provides a nucleic acid sequence that encodes the fusion protein of SEQ ID NO:6, including all embodiments and percent sequence identities described herein. In embodiments, the disclosure provides a nucleic acid sequence that encodes the fusion protein of SEQ ID NO:7, including all embodiments and percent sequence identities described herein.

In embodiments, the nucleic acids encode: (i) the fusion proteins described herein, including embodiments thereof, and (ii) connexin 43, including all embodiments thereof described herein. In embodiments, the nucleic acid sequence encodes: (i) a fusion protein described herein, including fusion proteins having amino acid sequences with certain percent sequence identities described herein, including embodiments thereof, and (ii) connexin 43, including embodiments thereof described herein. In embodiments, the nucleic acid is RNA. In embodiments, the nucleic acid is messenger RNA. In embodiments, the nucleic acid is DNA. In embodiments, the disclosure provides a nucleic acid sequence that encodes: (i) connexin 43, including all embodiments thereof, and (ii) a fusion protein comprising a zinc finger domain and a transcriptional activator, wherein the fusion protein is capable of binding to the promoter sequence of the cystic fibrosis transmembrane conductance regulator gene. In embodiments, the disclosure provides a nucleic acid sequence that encodes connexin 43, including all embodiments thereof, and the fusion protein of SEQ ID NO:2, including all embodiments and percent sequence identities described herein. In embodiments, the disclosure provides a nucleic acid sequence that encodes connexin 43, including all embodiments thereof, and the fusion protein of SEQ ID NO:3, including all embodiments and percent sequence identities described herein. In embodiments, the disclosure provides a nucleic acid sequence that encodes connexin 43, including all embodiments thereof, and the fusion protein of SEQ ID NO:4, including all embodiments and percent sequence identities described herein. In embodiments, the disclosure provides a nucleic acid sequence that encodes connexin 43, including all embodiments thereof, and the fusion protein of SEQ ID NO:5, including all embodiments and percent sequence identities described herein. In embodiments, the disclosure provides a nucleic acid sequence that encodes connexin 43, including all embodiments thereof, and the fusion protein of SEQ ID NO:6, including all embodiments and percent sequence identities described herein. In embodiments, the disclosure provides a nucleic acid sequence that encodes connexin 43, including all embodiments thereof, and the fusion protein of SEQ ID NO:7, including all embodiments and percent sequence identities described herein. In embodiments, connexin 43 comprises SEQ ID NO:18 or SEQ ID NO:19. In embodiments, connexin 43 comprises SEQ ID NO:18. In embodiments, connexin 43 comprises SEQ ID NO:19.

In embodiments, the disclosure provides a nucleic acid having SEQ ID NO:31. In embodiments, the nucleic acid has at least 80% sequence identity to SEQ ID NO:31. In embodiments, the nucleic acid has at least 85% sequence identity to SEQ ID NO:31. In embodiments, the nucleic acid has at least 90% sequence identity to SEQ ID NO:31. In embodiments, the nucleic acid has at least 92% sequence identity to SEQ ID NO:31. In embodiments, the nucleic acid has at least 94% sequence identity to SEQ ID NO:31. In embodiments, the nucleic acid has at least 95% sequence identity to SEQ ID NO:31. In embodiments, the nucleic acid has at least 96% sequence identity to SEQ ID NO:31. In embodiments, the nucleic acid has at least 98% sequence identity to SEQ ID NO:31. In embodiments, the nucleic acid that has SEQ ID NO:31.

Vectors

Provided herein are vectors, such as expression vectors, that comprise the nucleic acid sequences of the present disclosure (including all embodiments thereof), and which encode the fusion protein of the present disclosure (including all embodiments thereof). The vectors can be used to produce the fusion protein of the disclosures in exosomes and/or cells. In embodiments, the vectors comprise the nucleic acid sequences of the present disclosure (including all embodiments thereof) which encode connexin 43 (including all embodiments thereof). In embodiments, the vectors comprise the nucleic acid sequences of the present disclosure (including all embodiments thereof) which encode: (i) the fusion protein of the present disclosure (including all embodiments thereof), and (ii) connexin 43 (including all embodiments thereof). The vectors can be used to produce the fusion protein and connexin 43 in cells. The expression vector can be transfected into cells (e.g., eukaryotic cells such as mammalian cells or human cell lines or prokaryotic cells such as Escherichia coli) in which the fusion protein is expressed and the expressed peptide can be isolated and purified using various techniques available in the field. In embodiments, the vector is a lentiviral vector.

In embodiments, the expression vector is capable of directing the expression of nucleic acids to which they are operatively linked. The term “operably linked” means that the nucleotide sequence of interest is linked to regulatory sequence(s) in a manner that allows for expression of the nucleotide sequence. The regulatory sequence may include, for example, promoters, enhancers, and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors based on various viral sequences as well as those contemplated for eukaryotic target cells or prokaryotic target cells. The “target cells” may refer to the cells where the expression vector is transfected and the nucleotide sequence encoding the fusion protein is expressed. In embodiments, the target cells are the cells used for production of the fusion protein of the present disclosure for later use. Therefore, in these embodiments the expressed fusion protein are isolated from the target cells and administered to a subject later for a therapeutic purpose. Therefore, when the expression vector is transfected into such cells, the fusion protein expressed from the vector activates the viral transcription in the cells. Any vectors can be used so long as they are compatible with the desired or intended target cell. The skilled person in the art can use any suitable vectors known and available in the art depending on their system, e.g., the target cell or the process of culturing cell and purifying the fusion proteins.

In embodiments, a vector has one or more transcription and/or translation control elements. Depending on the target/vector system utilized, any of a number of suitable transcription and translation control elements, including constitutive and inducible promoters, transcription enhancer elements, transcription terminators, etc. can be used in the expression vector.

In embodiments, the vector is plasmid, a viral vector, a cosmid, or an artificial chromosome. In embodiments, the vector is a plasmid. In embodiments, the vector is a viral vector. In embodiments, the viral vector is a retroviral vector. In embodiments, the vector is a lentiviral vector.

Non-limiting examples of suitable eukaryotic promoters (i.e., promoters functional in a eukaryotic cell) include those from cytomegalovirus (CMV) immediate early, H1, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I. The promoter can be a constitutive promoter (e.g., CMV promoter, UBC promoter). In embodiments, the promoter can be a spatially restricted and/or temporally restricted promoter (e.g., a tissue specific promoter, a cell type specific promoter, etc.).

Exosomes

One means to deliver zinc finger proteins (ZFP) intracellularly is by cell-derived exosomes. The emerging field of extracellular particles and exosomes, collectively referred to here as exosomes, offers a unique, elegant opportunity to utilize basic cellular processes of exosome genesis to produce and deliver biologically relevant synthetic proteins. Exosomes are a subgroup of extracellular vesicles (EVs) originating in multivesicular bodies secreted into the extracellular environment after fusion with the plasma membrane (14, 15). Exosomes contain mRNAs, non-coding RNAs and proteins and transfer their contents from donor to recipient cells resulting in functional changes of target cells (16-18). Thus, exosomes are important mediators that facilitate intercellular communication without direct cell-to-cell contact (19). Among exosomes derived from various cellular origins, mesenchymal stem cell-derived exosomes have gained much attention for their potential to deliver diverse biomolecules (20) and modulate states of inflammation (21). Due to their inherent anti-inflammatory properties (21), and observations that inflammation is observed in CF (22), the inventors selected MSCs to produce engineered exosomes packaged with the CFTR activator CFZF-VPR. MSC-derived CFZF-VPR containing exosomes can functionally activate CFTR expression in both HEK293 cell lines and HuBECs. Collectively, the observations presented here demonstrate that exosomes carrying the transcriptional activator CFZF-VPR can target and functionally activate CFTR.

Provided here are exosomes comprising the fusion proteins, nucleic acids, and vectors described herein, including all embodiments thereof. In embodiments, the exosomes are mesenchymal stem cell exosomes, i.e., exosomes obtained from (derived from) mesenchymal stem cells. Thus, the disclosure provides mesenchymal stem cell exosomes comprising the fusion proteins, nucleic acids, and vectors described herein, including all embodiments thereof.

In embodiments, the exosomes comprise a fusion protein described herein, including all embodiments thereof. In embodiments, the exosomes comprise a fusion protein described herein, including all embodiments thereof, and connexin 43 described herein, including all embodiments thereof. In embodiments, the exosomes are mesenchymal stem cell exosomes.

In embodiments, the exosomes comprise a nucleic acid encoding the fusion protein described herein, including all embodiments thereof. In embodiments, the exosomes comprise a first nucleic acid encoding a fusion protein described herein, including all embodiments thereof, and a second nucleic acid encoding connexin 43 described herein, including all embodiments thereof. In embodiments, the exosomes comprise a nucleic acid encoding a fusion protein and connexin 43, as described herein, including all embodiments thereof. In embodiments, the exosomes are mesenchymal stem cell exosomes. In embodiments, the nucleic acid is RNA. In embodiments, the nucleic acid is mRNA.

In embodiments, the exosomes comprise a vector having a nucleic acid encoding the fusion protein described herein, including all embodiments thereof. In embodiments, the exosomes comprise a first vector having a nucleic acid encoding a fusion protein described herein, including all embodiments thereof, and a second vector having a nucleic acid encoding connexin 43 described herein, including all embodiments thereof. In embodiments, the exosomes comprise a vector having a nucleic acid encoding a fusion protein and connexin 43, as described herein, including all embodiments thereof. In embodiments, the exosomes are mesenchymal stem cell exosomes.

In embodiments, the exosomes comprise: (i) a fusion protein described herein, including all embodiments thereof; (ii) a nucleic acid encoding the fusion protein described herein, including all embodiments thereof; (iii) a vector having a nucleic acid encoding the fusion protein described herein, including all embodiments thereof; or (iv) a combination of two or more of the foregoing. In embodiments, the exosomes further comprise: (a) connexin 43, including all embodiments thereof; (b) a nucleic acid encoding connexin 43, including all embodiments thereof (where the nucleic acid is the same as or different from the nucleic acid encoding the fusion protein); (c) a vector having a nucleic acid encoding connexin 43, including all embodiments thereof (where the vector is the same as or different from the vector having the nucleic acid encoding the fusion protein); or (d) a combination of two or more of the foregoing. In embodiments, the exosomes are mesenchymal stem cell exosomes.

Cells

The disclosure provides cells comprising the fusion proteins, complexes, nucleic acids, vectors, exosomes, and pharmaceutical compositions described herein. Inside the cell, the fusion protein is as described herein, including embodiments thereof, perform transcriptional activation of the CFTR gene. In embodiments, the cell comprises a fusion protein described herein, including all embodiments thereof. In embodiments, the cell comprises a fusion protein described herein, including all embodiments thereof, and connexin 43 described herein, including all embodiments thereof. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

In embodiments, the cell comprises a complex described herein, including all embodiments thereof. In embodiments, the complex comprises a fusion protein described herein, including all embodiments thereof, bonded to a CFTR gene. In embodiments, the complex comprises a fusion protein described herein, including all embodiments thereof, bonded to a promoter sequence of a CFTR gene. In embodiments, the promoter sequence of the CFTR gene comprises SEQ ID NO:1. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

In embodiments, the cell comprises a nucleic acid encoding the fusion protein described herein, including all embodiments thereof. In embodiments, the cell comprises a first nucleic acid encoding a fusion protein described herein, including all embodiments thereof, and a second nucleic acid encoding connexin 43 described herein, including all embodiments thereof. In embodiments, the cell comprises a nucleic acid encoding a fusion protein and connexin 43, as described herein, including all embodiments thereof. In embodiments, the nucleic acid is RNA. In embodiments, the nucleic acid is mRNA. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

In embodiments, the cell comprises a vector having a nucleic acid encoding the fusion protein described herein, including all embodiments thereof. In embodiments, the cell comprises a first vector having a nucleic acid encoding a fusion protein described herein, including all embodiments thereof, and a second vector having a nucleic acid encoding connexin 43 described herein, including all embodiments thereof. In embodiments, the cell comprises a vector having a nucleic acid encoding a fusion protein and connexin 43, as described herein, including all embodiments thereof. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

In embodiments, the cell comprises an exosome which comprises a fusion protein described herein, including all embodiments thereof. In embodiments, the cell comprises an exosome which comprises a fusion protein described herein, including all embodiments thereof, and connexin 43 described herein, including all embodiments thereof. In embodiments, the cell comprises an exosome which comprises a nucleic acid encoding the fusion protein described herein, including all embodiments thereof. In embodiments, the cell comprises an exosome which comprises a first nucleic acid encoding a fusion protein described herein, including all embodiments thereof, and a second nucleic acid encoding connexin 43 described herein, including all embodiments thereof. In embodiments, the cell comprises an exosome which comprises a nucleic acid encoding a fusion protein and connexin 43, as described herein, including all embodiments thereof. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

In embodiments, the cell comprises an exosome which comprises a vector having a nucleic acid encoding the fusion protein described herein, including all embodiments thereof. In embodiments, the cell comprises an exosome which comprises a first vector having a nucleic acid encoding a fusion protein described herein, including all embodiments thereof, and a second vector having a nucleic acid encoding connexin 43 described herein, including all embodiments thereof. In embodiments, the cell comprises an exosome which comprises a vector having a nucleic acid encoding a fusion protein and connexin 43, as described herein, including all embodiments thereof. In embodiments, the cell is a mammalian cell. In embodiments, the cell is a mammalian cell. In embodiments, the cell is a human cell. In embodiments, the cell is a human epithelial cell. In embodiments, the cell is a human bronchial epithelial cell. In embodiments, the cell is a primary human bronchial epithelial cell. In embodiments, the cell is a human epithelial cell with a F508del mutation. In embodiments, the cell is a human bronchial epithelial cell with a F508del mutation. In embodiments, the cell is a primary human bronchial epithelial cell with a F508del mutation.

Pharmaceutical Compositions

Provided herein are pharmaceutical compositions comprising the active agents (e.g., fusion proteins, nucleic acids, vectors, exosomes) and a pharmaceutically acceptable excipient. The provided compositions are suitable for formulation and administration in vitro or in vivo. Suitable carriers and excipients and their formulations are described in Remington: The Science and Practice of Pharmacy, 21st Edition, David B. Troy, ed., Lippicott Williams & Wilkins (2005).

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the active agents (e.g., fusion proteins, nucleic acids, vectors, exosomes) can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 1 to about 99% of the weight of the unit. The amount of active agents (e.g., fusion proteins, nucleic acids, vectors, exosomes) in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for extremely rapid penetration, delivering high concentrations of the active agents to a small area.

The pharmaceutical compositions can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

Dose and Dosing Regimens

The dosage and frequency (single or multiple doses) of the active agents (e.g., fusion proteins, nucleic acids, vectors, exosomes) described herein, including all embodiments thereof, administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods described herein. Adjustment and manipulation of established dosages (e.g., frequency and duration) are well within the ability of those skilled in the art.

For any active agents described herein, the effective amount can be initially determined from cell culture assays. Target concentrations will be those concentrations of active agents that are capable of achieving the methods described herein, as measured using the methods described herein or known in the art. As is known in the art, effective amounts of active agents for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the active agents may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the active agents. Thereafter, the dosage is increased by small increments until the optimum effect under circumstances is reached. Dosage amounts and intervals can be adjusted individually to provide levels of the active agents effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active agents (e.g., fusion proteins, nucleic acids, vectors, exosomes) by considering factors such as compound potency, relative bioavailability, patient body weight, presence and severity of adverse side effects.

In embodiments, the active agent is administered to a patient at an amount of about 0.001 mg/kg to about 500 mg/kg. In embodiments, the active agent is administered to a patient in an amount of about 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the active agents. In embodiments, the active agent is administered to a patient in an amount from about 0.001 mg to about 1,000 mg per day, as a single dose, or in a dose administered two or three times per day.

Methods

The disclosure provides methods of treating cystic fibrosis in patients by administering an effective amount of the fusion proteins, nucleic acids, expression vectors, exosomes, cells, or pharmaceutical compositions described herein.

In embodiments, the disclosure provides methods of treating cystic fibrosis in a patient in need thereof by administering to the patient an effective amount of a fusion protein described herein, including all embodiments thereof. In embodiments, the disclosure provides methods of treating cystic fibrosis in a patient in need thereof by administering to the patient an effective amount of connexin 43 and a fusion protein described herein, including all embodiments thereof.

In embodiments, the disclosure provides methods of treating cystic fibrosis in a patient in need thereof by administering to the patient an effective amount of an exosome comprising the fusion protein described herein, including all embodiments thereof. In embodiments, the disclosure provides methods of treating cystic fibrosis in a patient in need thereof by administering to the patient an effective amount of an exosome comprising connexin 43 and the fusion protein described herein, including all embodiments thereof. In embodiments, the exosome is a mesenchymal stem cell exosome.

In embodiments, the disclosure provides methods of treating cystic fibrosis in a patient in need thereof by administering to the patient an effective amount of a nucleic acid described herein, including all embodiments thereof. In embodiments, the methods comprise administering an effective amount of a first nucleic acid that encodes the fusion proteins described herein and an effective amount of a second nucleic acid that encodes connexin 43. In embodiments, the methods comprise administering an effective amount of a nucleic acid that encodes connexin 43 and the fusion proteins described herein. In embodiments, the nucleic acid is RNA. In embodiments, the nucleic acid is mRNA.

In embodiments, the disclosure provides methods of treating cystic fibrosis in a patient in need thereof by administering to the patient an effective amount of an exosome comprising the nucleic acids described herein, including all embodiments thereof. In embodiments, the methods comprise administering an effective amount of an exosome comprising a first nucleic acid that encodes the fusion proteins described herein and a second nucleic acid that encodes connexin 43. In embodiments, the methods comprise administering an effective amount of an exosome comprising a nucleic acid that encodes connexin 43 and the fusion proteins described herein. In embodiments, the nucleic acid is RNA. In embodiments, the nucleic acid is mRNA.

In embodiments, the disclosure provides methods of treating cystic fibrosis in a patient in need thereof by administering to the patient an effective amount of a vector comprising a nucleic acid that encodes the fusion proteins described herein, including all embodiments thereof. In embodiments, the methods comprise administering to the patient an effective amount of a first vector comprising a nucleic acid that encodes the fusion proteins described herein and an effective amount of a second vector comprising a nucleic acid that encodes connexin 43. In embodiments, the methods comprise administering to the patient an effective amount of a vector comprising a nucleic acid that encodes connexin 43 and the fusion proteins described herein.

In embodiments, the disclosure provides methods of treating cystic fibrosis in a patient in need thereof by administering to the patient an effective amount of an exosome comprising the vector having a nucleic acid that encodes the fusion proteins described herein, including all embodiments thereof. In embodiments, the disclosure provides methods of treating cystic fibrosis in a patient in need thereof by administering to the patient an effective amount of an exosome comprising a first vector having a nucleic acid that encodes the fusion proteins described herein and a second vector having a nucleic acid that encodes connexin 43. In embodiments, the disclosure provides methods of treating cystic fibrosis in a patient in need thereof by administering to the patient an effective amount of an exosome comprising a vector having a nucleic acid that encodes connexin 43 and the fusion proteins described herein.

In embodiments, the disclosure provides methods of treating cystic fibrosis in a patient in need thereof by administering to the patient an effective amount of a cell described herein, including all embodiments thereof.

In embodiments, the disclosure provides methods of treating cystic fibrosis in a patient in need thereof by administering to the patient an effective amount of a pharmaceutical composition comprising the exosomes described herein, including all embodiments thereof.

In embodiments, the disclosure provides methods of treating cystic fibrosis in a patient in need thereof by (i) transducing cells ex vivo with the nucleic acid described herein, including embodiments thereof, to produce transduced cells; and (ii) administering an effective amount of the transduced cells to the patient, thereby treating cystic fibrosis. In embodiments, the cells are blood cells. In embodiments, the cells are peripheral blood mononuclear cells. In embodiments, the cells are autologous cells. In embodiments, the cells are allogenic cells. In embodiments, the methods further comprise obtaining cells from the patient prior to transduced the cells ex vivo. Methods for transduced cells ex vivo are known in the art. Methods for administering transduced cells to a patient are known in the art.

In embodiments, the disclosure provides methods of treating cystic fibrosis in a patient in need thereof by (i) transfecting cells ex vivo with the nucleic acid described herein, including embodiments thereof, to produce transfected cells; and (ii) administering an effective amount of the transfected cells to the patient, thereby treating cystic fibrosis. In embodiments, the cells are blood cells. In embodiments, the cells are peripheral blood mononuclear cells. In embodiments, the cells are autologous cells. In embodiments, the cells are allogenic cells. In embodiments, the methods further comprise obtaining cells from the patient prior to transfecting the cells ex vivo. Methods for transfecting cells ex vivo are known in the art. Methods for administering transfected cells to a patient are known in the art.

In embodiments, the disclosure provides methods of treating cystic of treating cystic fibrosis in a patient in need thereof by (i) transducing cells ex vivo with the vector of any one of claims 20 to 23 to produce transduced cells; and (ii) administering an effective amount of the transduced cells to the patient, thereby treating cystic fibrosis. In embodiments, the vector is a lentiviral vector. In embodiments, the cells are blood cells. In embodiments, the cells are peripheral blood mononuclear cells. In embodiments, the cells are autologous cells. In embodiments, the cells are allogenic cells. In embodiments, the methods further comprise obtaining cells from the patient prior to transducing the cells ex vivo. Methods for transducing cells ex vivo are known in the art. Methods for administering transduced cells to a patient are known in the art.

The disclosure provides methods of activating a CTFR gene by contacting an effective amount of the fusion proteins, nucleic acids, vectors, exosomes, or pharmaceutical compositions described herein with a CFTR gene. In embodiments, the disclosure provides methods of activating a CTFR gene by contacting a CFTR gene and an effective amount of a fusion protein described herein, including all embodiments thereof. In embodiments, the disclosure provides methods of activating a CTFR gene by contacting a CFTR gene and an effective amount of a nucleic acid described herein, including all embodiments thereof. In embodiments, the disclosure provides methods of activating a CTFR gene by contacting a CFTR gene and an effective amount of a vector described herein, including all embodiments thereof. In embodiments, the disclosure provides methods of activating a CTFR gene by contacting a CFTR gene and an effective amount of a exosome described herein, including all embodiments thereof. In embodiments, the disclosure provides methods of activating a CTFR gene by contacting a CFTR gene and an effective amount of a pharmaceutical composition described herein, including all embodiments thereof.

In the embodiments of treating cystic fibrosis described herein, the methods may further comprise administering to the patient an effective amount of a cystic fibrosis therapeutic agent. In embodiments, the cystic fibrosis therapeutic agent comprises an antibiotic, a mucus thinner, a CFTR modulator, or a combination of two or more thereof. In embodiments, the cystic fibrosis therapeutic agent comprises an antibiotic. In embodiments, the cystic fibrosis therapeutic agent comprises a mucus thinner. In embodiments, the cystic fibrosis therapeutic agent comprises a CFTR modulator. In embodiments, the cystic fibrosis therapeutic agent comprises an antibiotic and a mucus thinner. In embodiments, the cystic fibrosis therapeutic agent comprises an antibiotic and a CFTR modulator. In embodiments, the cystic fibrosis therapeutic agent comprises a mucus thinner and a CFTR modulator. In embodiments, the cystic fibrosis therapeutic agent comprises an antibiotic, a mucus thinner, and a CFTR modulator. In embodiments, the CFTR modulator comprises ivacaftor, lumacaftor, tezacaftor, elexacaftor, or a combination of two or more thereof. In embodiments, the CFTR modulator comprises ivacaftor.

In embodiments, the CFTR modulator comprises lumacaftor. In embodiments, the CFTR modulator comprises ivacaftor and lumacaftor. In embodiments, the CFTR modulator comprises tezacaftor. In embodiments, the CFTR modulator comprises ivacaftor and tezacaftor. In embodiments, the CFTR modulator comprises elexacaftor. In embodiments, the CFTR modulator comprises ivacaftor, tezacaftor, and elexacaftor. In embodiments, the mucus thinner comprises hypertonic saline, domase alfa, or a combination thereof. In embodiments, the mucus thinner comprises hypertonic saline and domase alfa. In embodiments, the mucus thinner comprises hypertonic saline. In embodiments, the mucus thinner comprises domase alfa. In embodiments, the antibiotic is any antibiotic useful for treating infections caused by Staphylococcus aureus, methicillin-resistant Staphylococcus aureus, Pseudomonas aeruginosa, Mycobacterium abscessus, Mycobacterium avium complex, Burkholderia cepacia, Stenotrophomonas maltophilia, Haemophilus influenze, Achromobacter xylosoxidans, or a combination thereof. In embodiments, the antibiotic is levofloxacin.

The terms “treating” or “treatment” include any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. “Treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. In other words, “treatment” as used herein includes any cure, amelioration, or prevention of a disease.

“Treating” or “treatment” as used herein includes prophylactic treatment. Treatment may prevent the disease from occurring; inhibit the disease's spread; relieve the disease's symptoms, fully or partially remove the disease's underlying cause, shorten a disease's duration, or do a combination of these things. The term “treating” and conjugations thereof may include prevention of an injury, pathology, condition, or disease. In embodiments, treating is preventing. In embodiments, treating does not include preventing. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by diagnostic assays (e.g., assays described herein or known in the art). In embodiments, chronic administration may be required. For example, the compositions are administered to the subject in an amount and for a duration sufficient to treat the patient.

The term “prevent” refers to a decrease in the occurrence of disease symptoms in a patient. The prevention may be complete (no detectable symptoms) or partial, such that fewer symptoms are observed than would likely occur absent treatment.

The term “patient” or “subject” refers to a living organism suffering from or prone to a disease or condition that can be treated by administration of a pharmaceutical composition. Non-limiting examples include humans, other mammals, bovines, rats, mice, dogs, monkeys, and other non-mammalian animals. In embodiments, a subject is human.

A “effective amount” is an amount sufficient for a compound of the disclosure to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent (e.g., compounds, hybridized nucleic acids) sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

For any compound described herein, the therapeutically effective amount can be initially determined from cell culture assays. Target concentrations will be those concentrations of active compound (e.g., fusion proteins, nucleic acids, vectors, exosomes) that are capable of achieving the methods described herein, as measured using the methods described herein or known in the art.

As is known in the art, therapeutically effective amounts for use in humans can also be determined from animal models. For example, a dose for humans can be formulated to achieve a concentration that has been found to be effective in animals. The dosage in humans can be adjusted by monitoring the effectiveness of the compositions, neural stem cells, and vesicles described herein, and adjusting the dosage upwards or downwards. Adjusting the dose to achieve maximal efficacy in humans based on the methods described above and other methods is well within the capabilities of the ordinarily skilled artisan.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. In embodiments, the neural stem cells, vesicles or pharmaceutical compositions described herein are parenterally administered to a patient. Other modes of delivery include, but are not limited to, the use of liposomal formulations, intravenous infusion, transdermal patches, etc. In embodiments, the administering does not include administration of any active agent other than the recited active agent.

Kits

Provided here are kits comprising the pharmaceutical compositions, exosomes, vectors, nucleic acids, and fusion proteins described herein. As part of the kit, materials and instruction are provided for both the activation of the CFTR gene or for the treatment of a patient with cystic fibrosis e.g., reagents or reaction mixtures for storage and use of kit components. In embodiments, the kit can contain one or more of the following components: (1) any of the fusion proteins in the present disclosure; (2) any of the nucleic acid sequences disclosed herein which encodes any of the fusion proteins; (3) any of the expression vectors disclosed herein which expresses any of the fusion proteins; (4) any of the exosomes described herein; (5); or any of the pharmaceutical compositions described herein; and instructions for how to use the kit components.

### EMBODIMENTS

Embodiment 1. A fusion protein comprising a zinc finger domain; wherein the zinc finger domain has at least 90% sequence identity to SEQ ID NO:8; and wherein the fusion protein is capable of binding to the promoter sequence of a cystic fibrosis transmembrane conductance regulator gene.
Embodiment 2. The fusion protein of Embodiment 1 having at least 95% sequence identity to SEQ ID NO:8.
Embodiment 3. The fusion protein of Embodiment 2 having SEQ ID NO:8.
Embodiment 4. The fusion protein of any one of Embodiments 1 to 3, wherein the fusion protein further comprises a transcriptional activator.
Embodiment 5. A fusion protein comprising a zinc finger domain and a transcriptional activator, wherein the fusion protein is capable of binding to the promoter sequence of the cystic fibrosis transmembrane conductance regulator gene, wherein the promoter sequence has at least 95% sequence identity to SEQ ID NO:1.
Embodiment 6. The fusion protein of Embodiment 5, wherein the promoter comprises SEQ ID NO:1.
Embodiment 7. The fusion protein of Embodiment 5 or 6, wherein the zinc finger domain has at least 95% sequence identity to SEQ ID NO:8.
Embodiment 8. The fusion protein of Embodiment 7 having SEQ ID NO:8.
Embodiment 9. A fusion protein comprising, from N-terminus to C-terminus, a zinc finger domain having at least 90% sequence identity to SEQ ID NO:8, VP64 having at least 90% sequence identity to SEQ ID NO:9, p65 having at least 90% sequence identity to SEQ ID NO:10, and Rta having at least 90% sequence identity to SEQ ID NO:11.
Embodiment 10. The fusion protein of Embodiment 9, wherein the zinc finger domain has at least 95% sequence identity to SEQ ID NO:8, VP64 has at least 95% sequence identity to SEQ ID NO:9, p65 has at least 95% sequence identity to SEQ ID NO:10, and Rta has at least 95% sequence identity to SEQ ID NO:11.
Embodiment 11. The fusion protein of Embodiment 10, wherein the zinc finger domain having SEQ ID NO:8, VP64 having SEQ ID NO:9, p65 having SEQ ID NO:10, and Rta having SEQ ID NO:11.
Embodiment 12. The fusion protein of any one of Embodiments 9 to 11, further comprising one or more glycine-serine linkers.
Embodiment 13. The fusion protein of any one of Embodiments 9 to 12, further comprising a nuclear localization signal, a TAT domain, an Myc tag, or a combination of two or more thereof.
Embodiment 14. The fusion protein of Embodiment 13, wherein the nuclear localization signal comprises SEQ ID NO:12 or SEQ ID NO:13; the TAT domain comprises SEQ ID NO:14 or SEQ ID NO:15; and the Myc tag comprises SEQ ID NO:16.
Embodiment 15. A fusion protein having at least 90% sequence identity to SEQ ID NO:3, SEQ ID NO:4, SEQ ID NO:5, SEQ ID NO:6, SEQ ID NO:7, or SEQ ID NO:2.
Embodiment 16. The fusion protein of Embodiment 15 having at least 95% sequence identity to SEQ ID NO:3, SEQ ID NO:4, SEQ ID NO:5, SEQ ID NO:6, SEQ ID NO:7, or SEQ ID NO:2.
Embodiment 17. The fusion protein of Embodiment 16 having SEQ ID NO:3, SEQ ID NO:4, SEQ ID NO:5, SEQ ID NO:6, SEQ ID NO:7, or SEQ ID NO:2.
Embodiment 18. A complex comprising the fusion protein of any one of Embodiments 1 to 17 bonded to a promoter sequence of a cystic fibrosis transmembrane conductance regulator gene.
Embodiment 19. The complex of Embodiment 18, wherein the promoter sequence has at least 95% sequence identity to SEQ ID NO:1.
Embodiment 20. The complex of Embodiment 19, wherein the promoter sequence comprises SEQ ID NO:1.
Embodiment 21. The complex of any one of Embodiments 18 to 20, wherein the cystic fibrosis transmembrane conductance regulator gene has a deletion of a phenylalanine residue at position 508 (F508del).
Embodiment 22. A nucleic acid encoding the fusion protein of any one of Embodiments 1 to 17.
Embodiment 23. The nucleic acid of Embodiment 22, further encoding a connexin 43 peptide.
Embodiment 24. The nucleic acid of Embodiment 23, wherein the connexin 43 peptide comprises SEQ ID NO:18 or SEQ ID NO:19.
Embodiment 25. The nucleic acid of any one of Embodiments 22 to 24, wherein the nucleic acid is mRNA.
Embodiment 26. A vector comprising a nucleic acid encoding the fusion protein of any one of Embodiments 1 to 17.
Embodiment 27. The vector of Embodiment 26, further comprising a nucleic acid encoding a connexin 43 peptide.
Embodiment 28. The vector of Embodiment 27, wherein the connexin 43 peptide comprises SEQ ID NO:18 or SEQ ID NO:19.
Embodiment 29. The vector of any one of Embodiments 26 to 28, wherein the vector is a lentiviral vector.
Embodiment 30. An exosome comprising the fusion protein of any one of Embodiments 1 to 17.
Embodiment 31. The exosome of Embodiment 30, further comprising (a) a connexin 43 peptide; (b) a nucleic acid encoding a connexin 43 peptide; or (c) a vector comprising a nucleic acid encoding a connexin 43 peptide.
Embodiment 32. The exosome of Embodiment 31, wherein the connexin 43 peptide comprises SEQ ID NO:18 or SEQ ID NO:19.
Embodiment 33. An exosome comprising the nucleic acid of any one of Embodiments 22 to 25.
Embodiment 34. The exosome of Embodiment 27, further comprising (a) a connexin 43 peptide; (b) a nucleic acid encoding a connexin 43 peptide; or (c) a vector comprising a nucleic acid encoding a connexin 43 peptide.
Embodiment 35. The exosome of Embodiment 34, wherein the connexin 43 peptide comprises SEQ ID NO:18 or SEQ ID NO:19.
Embodiment 36. An exosome comprising the fusion protein of any one of Embodiments 1 to 17 and the nucleic acid of any one of Embodiments 22 to 25.
Embodiment 37. The exosome of Embodiment 36, further comprising a connexin 43 peptide, a nucleic acid encoding a connexin 43 peptide, or a combination thereof.
Embodiment 38. The exosome of Embodiment 37, wherein the connexin 43 peptide comprises SEQ ID NO:18 or SEQ ID NO:19.
Embodiment 39. An exosome comprising the vector of any one of Embodiments 26 to 29.
Embodiment 40. The exosome of Embodiment 39, further comprising (a) a connexin 43 peptide; (b) a nucleic acid encoding a connexin 43 peptide; or (c) a vector comprising a nucleic acid encoding a connexin 43 peptide.
Embodiment 41. The exosome of Embodiment 40, wherein the connexin 43 peptide comprises SEQ ID NO:18 or SEQ ID NO:19.
Embodiment 42. The exosome of any one of Embodiments 30 to 41, wherein the exosome is a mesenchymal stem cell exosome.
Embodiment 43. A cell comprising the fusion protein of any one of Embodiments 1 to 17.
Embodiment 44. A cell comprising the nucleic acid of any one of Embodiments 22 to 25.
Embodiment 45. A cell comprising the vector of any one of Embodiments 26 to 29.
Embodiment 46. A cell comprising the exosome of any one of Embodiments 30 to 42.
Embodiment 47. A cell comprising the complex of any one of Embodiments 18 to 21.
Embodiment 48. A pharmaceutical composition comprising the fusion protein of any one of Embodiments 1 to 17, and a pharmaceutically acceptable excipient.
Embodiment 49. A pharmaceutical composition comprising the nucleic acid of any one of Embodiments 22 to 25, and a pharmaceutically acceptable excipient.
Embodiment 50. A pharmaceutical composition comprising the vector of any one of Embodiments 26 to 29, and a pharmaceutically acceptable excipient.
Embodiment 51. A pharmaceutical composition comprising the exosome of any one of Embodiments 30 to 42, and a pharmaceutically acceptable excipient.
Embodiment 52. A pharmaceutical composition comprising the cell of any one of Embodiments 43 to 47, and a pharmaceutically acceptable excipient.
Embodiment 53. A method of treating cystic fibrosis in a patient in need thereof, the method comprising administering to the patient an effective amount of the fusion protein of any one of Embodiments 1 to 17.
Embodiment 54. A method of treating cystic fibrosis in a patient in need thereof, the method comprising administering to the patient an effective amount of the nucleic acid of any one of Embodiments 22 to 25.
Embodiment 55. A method of treating cystic fibrosis in a patient in need thereof, the method comprising administering to the patient an effective amount of the vector of any one of Embodiments 26 to 29.
Embodiment 56. A method of treating cystic fibrosis in a patient in need thereof, the method comprising administering to the patient an effective amount of the exosome of any one of Embodiments 30 to 42.
Embodiment 57. A method of treating cystic fibrosis in a patient in need thereof, the method comprising administering to the patient an effective amount of the pharmaceutical composition of any one of Embodiments 48 to 52.
Embodiment 58. A method of treating cystic fibrosis in a patient in need thereof, the method comprising: (i) transducing cells ex vivo with the nucleic acid of any one of Embodiments 22 to 25 to produce transduced cells; and (ii) administering an effective amount of the transduced cells to the patient, thereby treating cystic fibrosis.
Embodiment 59. A method of treating cystic fibrosis in a patient in need thereof, the method comprising: (i) transfecting cells ex vivo with the nucleic acid of any one of Embodiments 22 to 25 to produce transfected cells; and (ii) administering an effective amount of the transfected cells to the patient, thereby treating cystic fibrosis.
Embodiment 60. The method of Embodiment 58 or 59, wherein the cells are peripheral blood mononuclear cells.
Embodiment 61. The method of any one of Embodiments 58 to 60, wherein the cells are autologous cells.
Embodiment 62. The method of any one of Embodiments 58 to 61, further comprising obtaining cells from the patient prior to transfecting the cells ex vivo.
Embodiment 63. A method of treating cystic fibrosis in a patient in need thereof, the method comprising: (i) transducing cells ex vivo with the vector of any one of Embodiments 26 to 29 to produce transduced cells; and (ii) administering an effective amount of the transduced cells to the patient, thereby treating cystic fibrosis.
Embodiment 64. The method of Embodiment 63, wherein the cells are peripheral blood mononuclear cells.
Embodiment 65. The method of Embodiment 63 or 64, wherein the cells are autologous cells.
Embodiment 66. The method of any one of Embodiments 63 to 65, further comprising obtaining cells from the patient prior to transducing the cells ex vivo.
Embodiment 67. The method of any one of Embodiments 53 to 66, wherein the patient has a deletion of a phenylalanine residue at position 508 (F508del) in the cystic fibrosis transmembrane conductance regulator gene.
Embodiment 68. The method of any one of Embodiments 53 to 67, further comprising administering to the patient an effective amount of a cystic fibrosis therapeutic agent.
Embodiment 69. The method of Embodiment 68, wherein the cystic fibrosis therapeutic agent is an antibiotic, a mucus thinner, a CFTR modulator, or a combination of two or more thereof.
Embodiment 70. A method of activating transcription in a cystic fibrosis transmembrane conductance regulator gene, the method comprising contacting an effective amount of the fusion protein of any one of Embodiments 1 to 17 with the cystic fibrosis transmembrane conductance regulator gene.
Embodiment 71. The method of Embodiment 70, comprising contacting the effective amount of the fusion protein with the promoter sequence of the cystic fibrosis transmembrane conductance regulator gene.
Embodiment 72. A zinc finger domain having at least 90% sequence identity to SEQ ID NO:8.
Embodiment 73. The zinc finger domain of Embodiment 72 having at least 95% sequence identity to SEQ ID NO:8.
Embodiment 74. The zinc finger domain of Embodiment 73 having SEQ ID NO:8.
Embodiment 75. The zinc finger domain of any one of Embodiments 72 to 74 that is capable of binding to the promoter sequence of a cystic fibrosis transmembrane conductance regulator gene.
Embodiment 76. The zinc finger domain of Embodiment 75, wherein the promoter sequence of the cystic fibrosis transmembrane conductance regulator gene has at least 95% sequence identity to SEQ ID NO:1.
Embodiment 77. The zinc finger domain of Embodiment 76, wherein the promoter sequence of the cystic fibrosis transmembrane conductance regulator gene has SEQ ID NO:1.

### EXAMPLES

In the examples herein, the CFZF-VPR fusion protein is SEQ ID NO:3, and the flag-tagged version of the CFZF-VPR fusion protein is SEQ ID NO:2.

Enhanced CFTR expression in HuBECs by the CFZF-VPR.

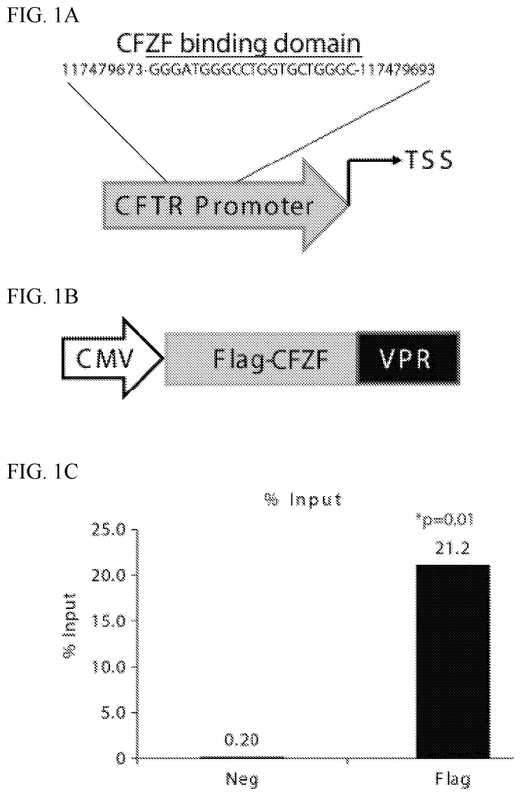

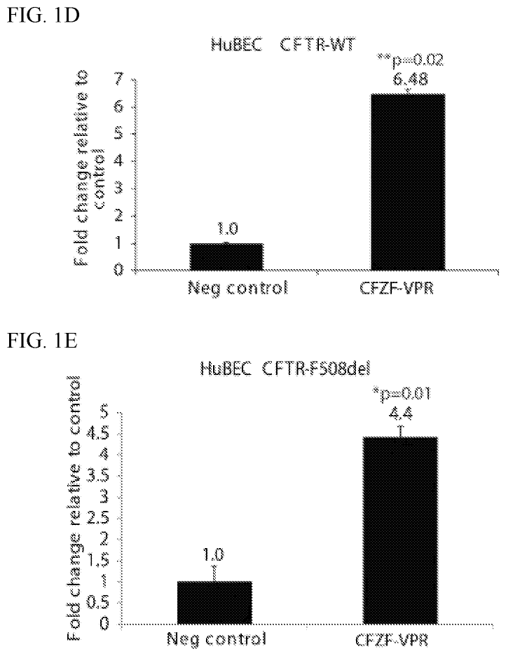

We investigated a synthetic zinc finger protein fused to VP64 (Herpesvirus transcription factor), p65 (NFκB subunit), and Rta (an activator of Epstein-Barr virus genes)(VPR)(11) to specifically activate the CFTR gene. The zinc finger protein (CFZF), targeted to the CFTR promoter, was fused to VPR (13) to generate CFZF-VPR (FIG. 1A, Table 1). To test whether CFZF-VPR binds directly to the CFTR promoter, a FLAG-tag ZFP-VPR vector was constructed with a CMV promoter used to express the transgene (FIG. 1B), and used in a chromatin immunoprecipitation assay (23). ChIP analysis verified that the CFZF-VPR is enriched at the CFTR promoter relative to the non-transfected HuBEC used as negative controls (FIG. 1C). Next, we tested whether the CFZF-VPR was able to activate mutant CFTR transcription. Both wild-type (WT) and the F508del-CFTR HuBECs were found to exhibit significant transcriptional activation of CFTR following transfection with CFZF-VPR (FIGS. 1D-1E), demonstrating that CFZF-VPR is a transcriptional activator of CFTR which can activate both WT and F508del-CFTR variants. Collectively, these data demonstrate the CFZF-VPR binds the CFTR promoter and activates transcription.

Mesenchymal Stem Cell CFZP-VPR packaged exosomes activate CFTR expression in target cells.

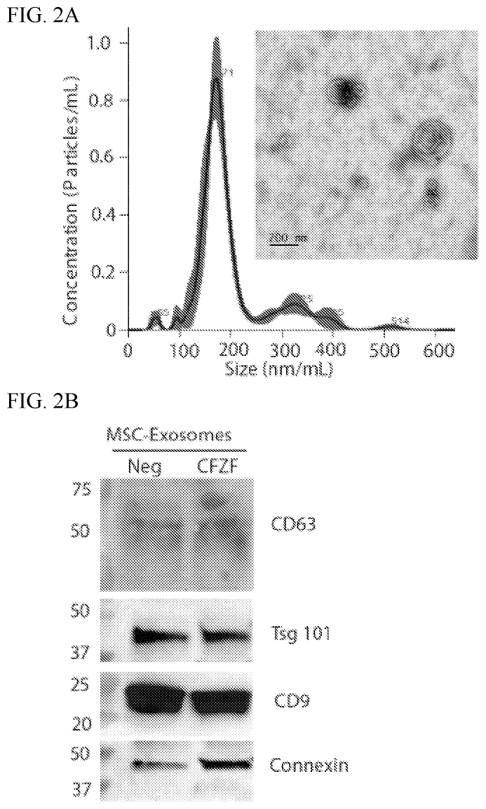

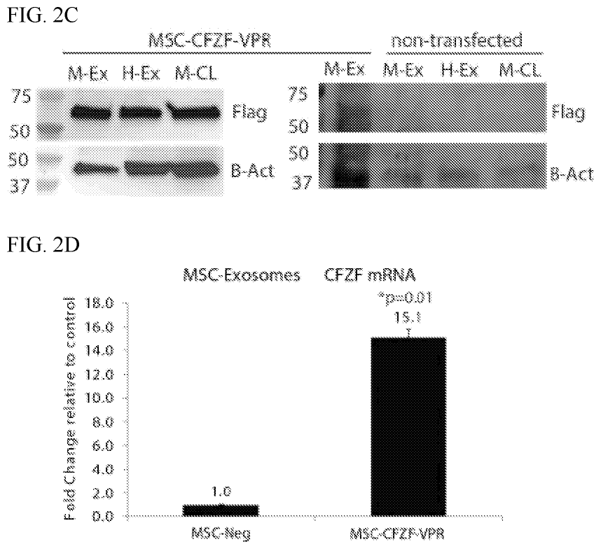

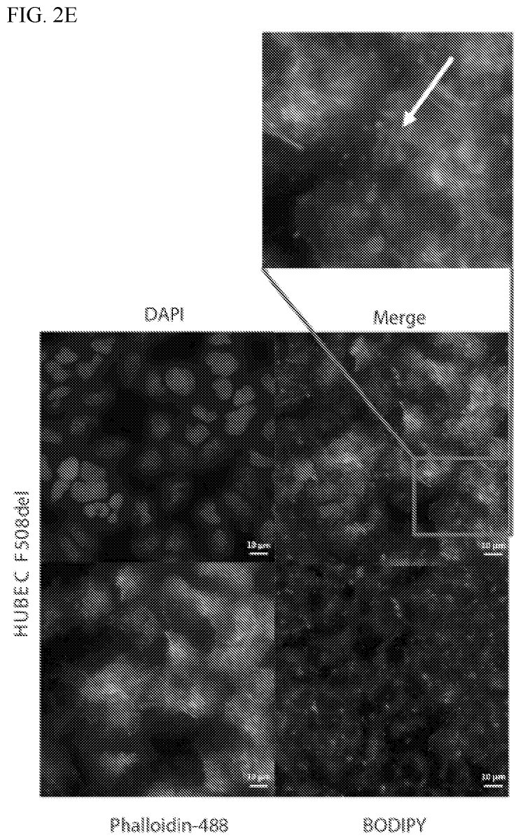

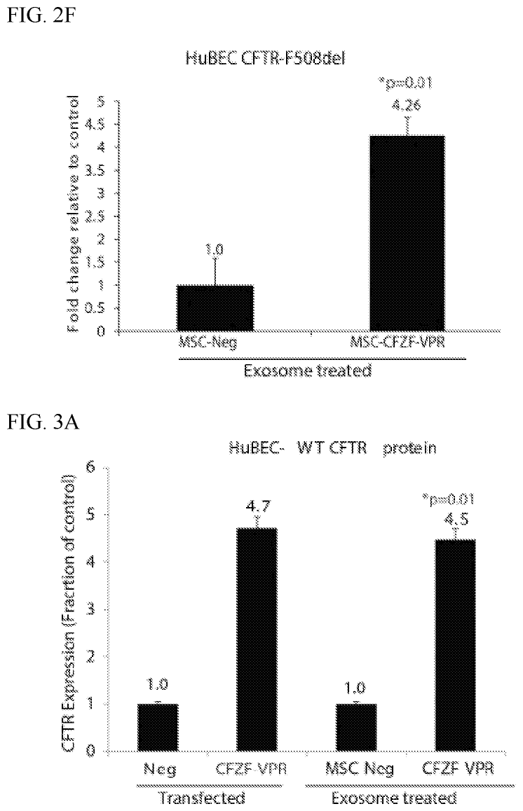

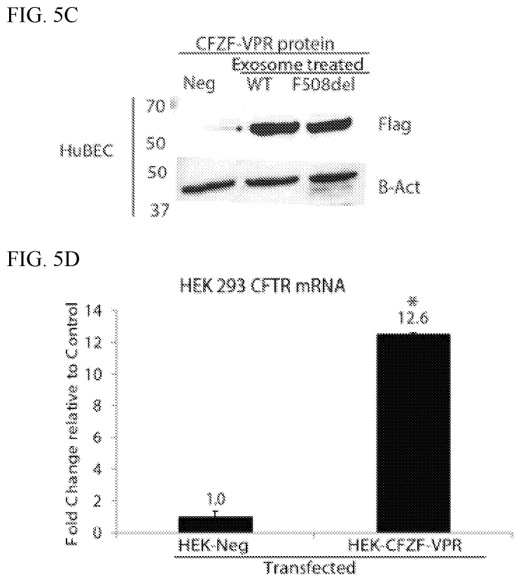

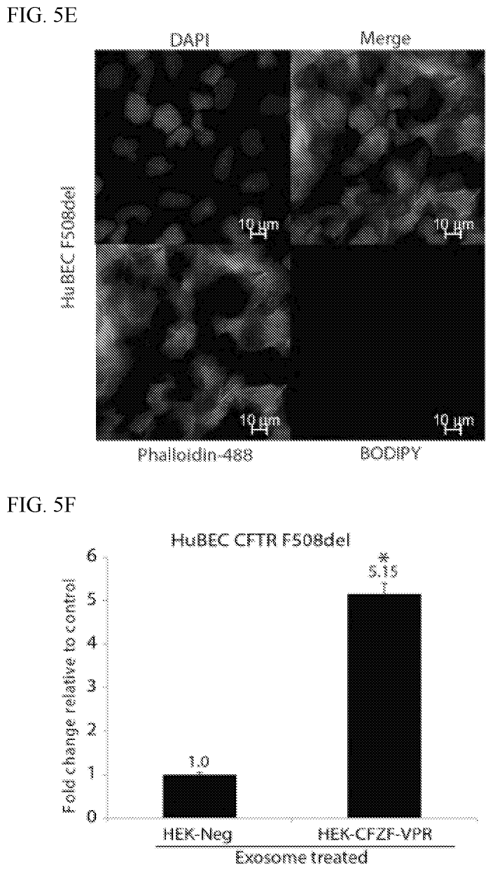

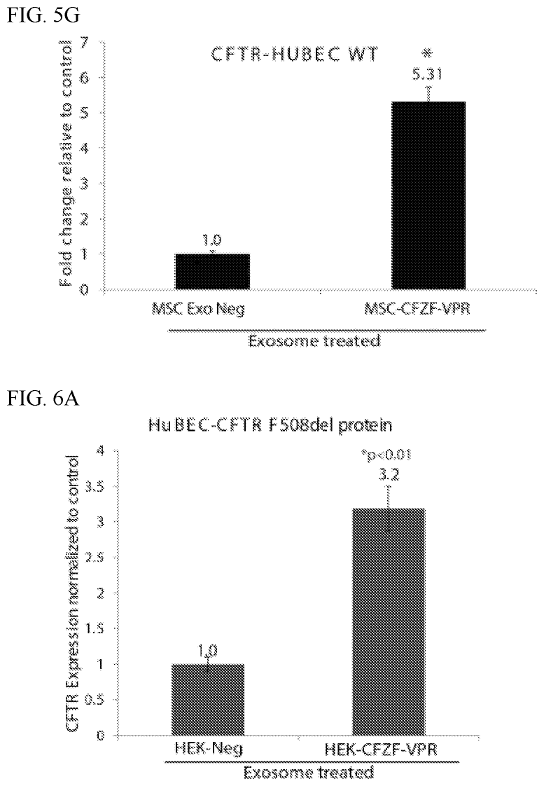

Exosomes are promising tools for drug delivery (24). Here, we developed exosomes carrying CFZF-VPR using a cell producer system by co-transfecting plasmids encoding the CFZP-VPR and Connexin 43 (Cx43) (SEQ ID NO:19), to facilitate endosomal release of payloads in target cells (25). Exosomes were produced and isolated from both CFZF-VPR/Cx43 transfected MSCs and HEK293 cells cultured in exosome-depleted FBS medium and from non-transfected cells which served as the negative control. The purified MSC-CFZF-VPR (FIG. 2A) and HEK293 CFZF-VPR exosomes (FIG. 5A) were characterized for their size and form using nanoparticle tracking analysis (NTA) and TEM analysis with the majority of particles ranging between about 150-170 nm in size. Importantly, exosomes from MSC or HEK293-cells transfected with Cx43 had an increased amount of protein detected as well as several other known extracellular vesicles markers (CD63, CD9 and TSG101) (FIG. 23, FIGS. 5B-5C).

Furthermore, FLAG-tagged CFZF-VPR protein (SEQ ID NO:2) was observed in both MSC-CFZF-VPR and HEK-CFZF-VPR exosomes and from MSC lysate (M-CL), but not in non-transfected control samples (FIG. 2C, FIG. 5B). CFZF-VPR mRNA was also found to be packaged in both MSC and HEK293 derived exosomes (FIG. 2D and FIG. 5D, respectively). Additionally, we demonstrated exosome uptake by adding Bodipy-labeled MSC-derived exosomes into the F508del-CFTR-HuBEC cells (FIG. 2E) and the CFZF-VPR protein was detected in the recipient HuBECs treated with MSC-CFZF-VPR compared with non-exosome treated (neg) HuBEC cells (FIG. 5B).

Next, we assessed the capacity of CFZF-VPR containing exosomes to activate CFTR expression. We observed a significant increase in CFTR mRNA expression 48 hours after treatment with either MSC or HEK293 derived CFZF-VPR exosomes in HuBECs with the F508del-CFTR mutation (FIG. 2F and FIG. 5F, respectively) and in WT-CFTR HuBECs treated with MSC-CFZF-VPR (FIG. 5G). These observations demonstrate that CFZF-VPR delivered by exosomes activate CFTR expression.

MSC-CFZF-VPR enhances CFTR protein expression and functionally improves chloride transport.

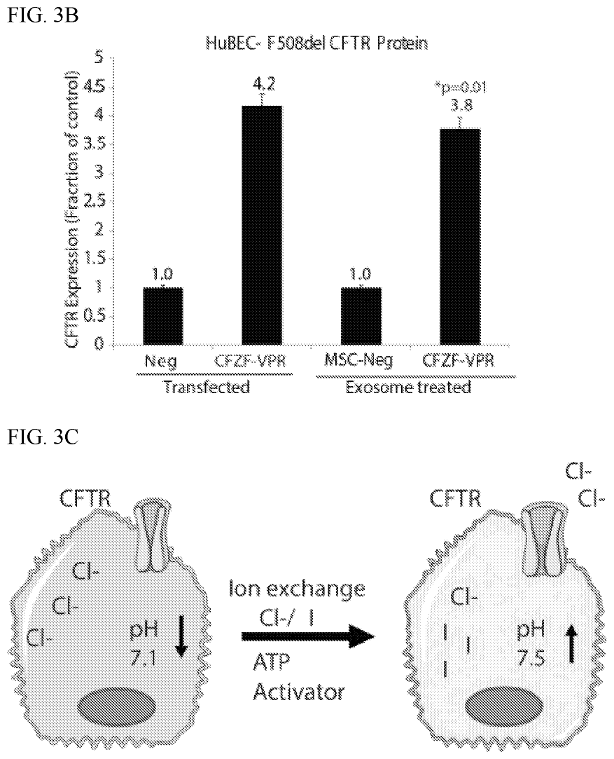

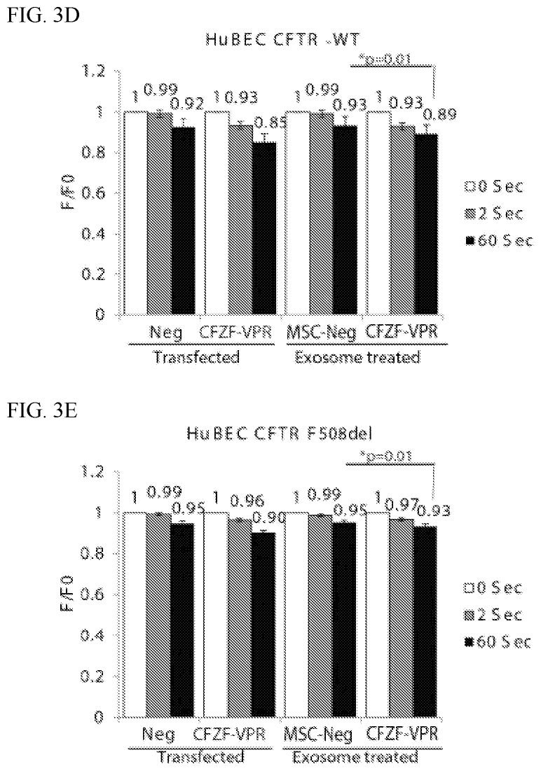

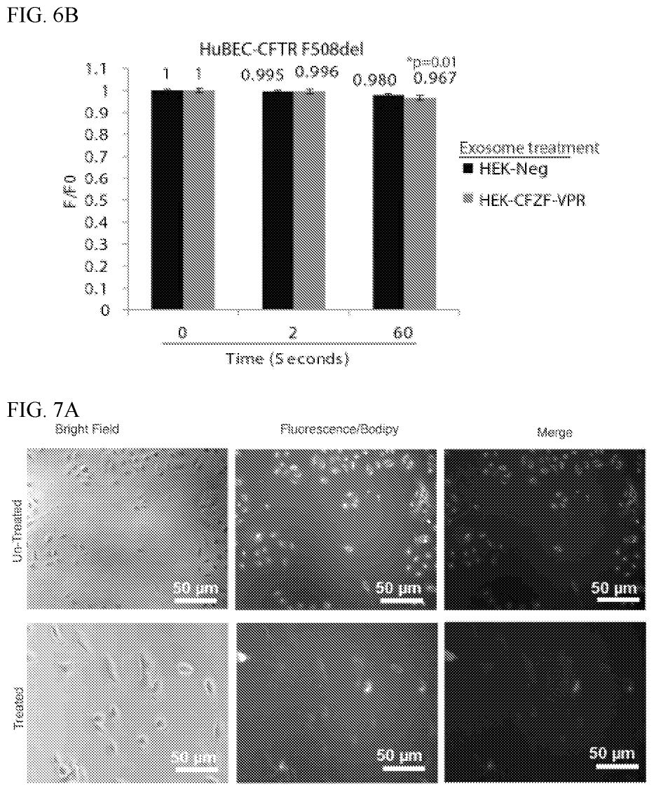

To investigate the effect of the MSC-derived exosomes containing CFZF-VPR in modulating the expression of CFTR gene we evaluated the CFTR protein expression in differentially treated cells. We found that treatment of both HuBEC-WT and HuBEC-F508del cells with the CFZF-VPR containing exosomes resulted in a significant increase in CFTR protein compared to those cells treated with control exosomes, and at equivalent levels of activation to plasmid transfection (FIGS. 3A-B). These data demonstrate that MSC-CFZF-VPR exosomes can induce F508del-CFTR protein expression. Notably, we also detected an increase in the CFTR protein in HuBEC-F508del cells treated with HEK293-derived exosomes carrying the CFZF-VPR (FIG. 6A), indicating that the observed exosome-mediated activation of CFTR is independent of the parent producer cells.

To determine if the observed increased CFTR protein expression was functionally relevant, we measured CFTR membrane activity using a halide assay which evaluates the increase of ion transport within cells expressing iodide-sensitive yellow fluorescent protein (YFP) (30), by using forskolin stimulation of CFTR and replacing chloride ions with iodide in cells expressing mutated YFP (FIG. 3C). To assess if CFZF-VPR containing exosomes functionally corrected the CFTR anion channel defect in vitro we evaluated the changes in YFP fluorescence in HuBECs pre-incubated with CFTR activator forskolin, treated with MSC-CFZF-VPR or controls in the presence of the potentiator (VX770) and the corrector (VX809) (33, 34). In both, wild-type and F508del HuBECs transfected with CFZF-VPR plasmid or treated with MSC exosomes containing CFZF-VPR (SEQ ID NO:3), we observed a significant decrease in fluorescence when compared to those cells treated with control exosomes (FIGS. 3D-3E, respectively). An reduction of fluorescence was also observed in HuBEC F508del treated with HEK293 exosomes containing CFZF-VPR (FIG. 6B). These observations demonstrate that MSC and HEK293 engineered exosomes functionally deliver a CFZF-VPR to primary CFTR-F508del bronchial epithelial cells to correct the anion channel defect in vitro.

### DISCUSSION

Herein, we designed a zinc finger protein activator, CFZF-VPR, targeting the CFTR promoter and demonstrated the successful induction of robust CFTR expression in primary human bronchial epithelial cells with the F508del mutation. These observations demonstrate that CFZF-VPR induced endogenous CFTR gene expression is useful to treat CF and that this approach is largely independent of the dominant F508del mutation found in the CFTR gene.

Exosomes have proven to be stable, biocompatible modalities to deliver a specific protein, miRNA or drug (28) and represents a valuable tool to deliver therapies to tissue in otherwise difficult to treat diseases such as CF. The incorporation of the CFZF-VPR mRNAs into the EVs from the parent cells can occur based on the lipid-mediated RNA loading mechanism. The inventors demonstrated that these exosomes delivered functional CFZF-VPR which rescued CFTR function in HuBECs with F508del mutation by increasing the levels of CFTR protein expression and function.

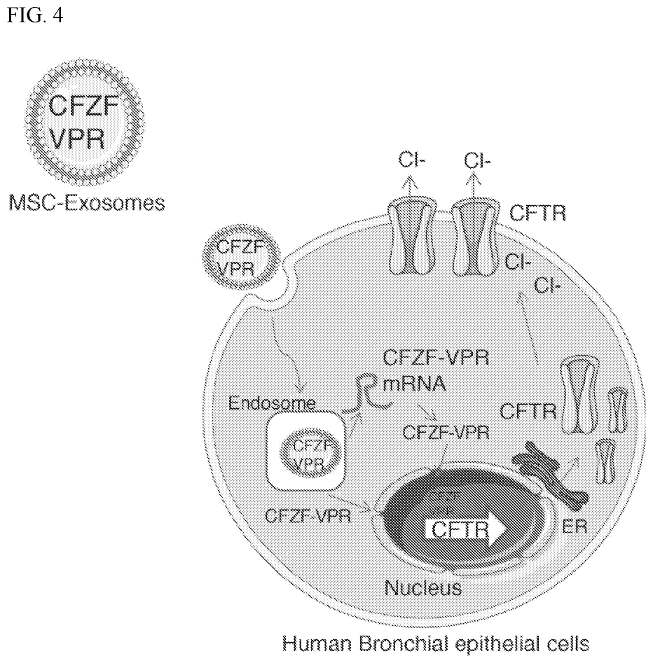

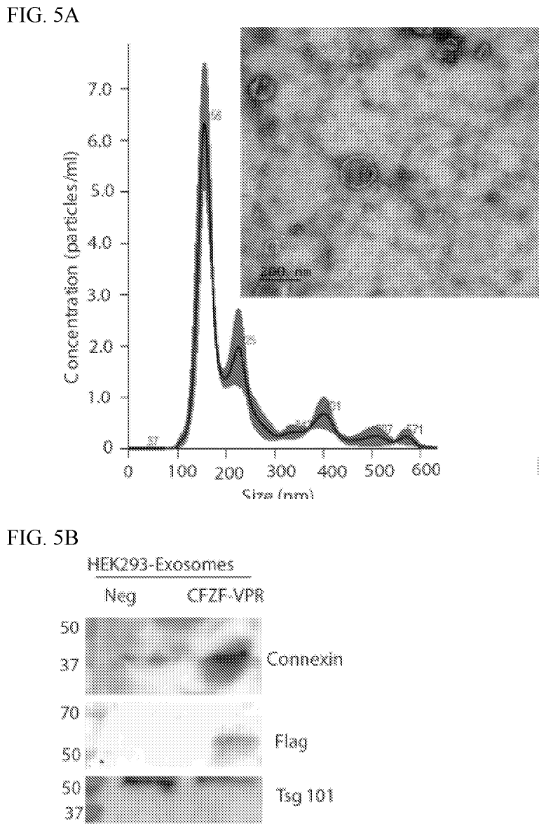

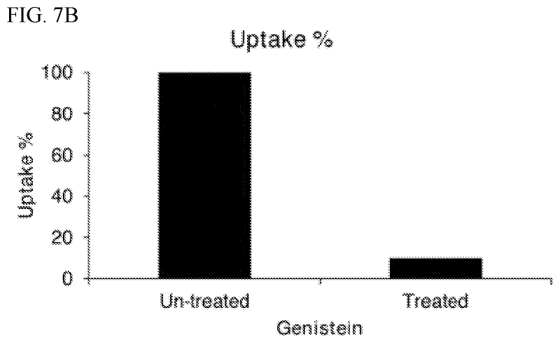

We also find here that the overexpression of Cx43 in the MSC and HEK293 exosome producer cells was found to significantly accentuate transfer of information, both CFZF-VPR RNA and protein from exosomes to target cells. This observation is noteworthy as it indicates that mutant Cx43 (S368A) over-expression is a useful delivery helper and collectively with CFZF-VPR proves to be a novel means to deliver functional therapeutic zinc finger activators to treat CF patients (FIG. 4). The presence of Cx43, a gap junction protein that links adjacent cells, on the exosome membrane surface facilitates the direct delivery of exosome cargo to recipient cells by interacting and facilitating the fusion of EV surface proteins with the target cell membrane (35, 36). Functionally, the mutant Cx43 incorporation is expected to inhibit serine phosphorylation, an action that is presumed to facilitate the formation of a hemichannel that might accentuate the delivery of proteins into the recipient cells (37)(FIG. 4). Supporting this notion, we find using endocytosis inhibitor genistein that there is a inhibition of the MSC derived CFZF-VPR exosome uptake in HuBECs (FIG. 7). Without intending to be bound by any theory, these data indicate that MSC-derived exosomes loaded with CFZF-VPR might be internalized in the cells through a clathrin-independent endocytic mechanism. However, to what extent this molecular pathway was engaged in the observations presented here remains to be determined.

Collectively, the results presented here represent the first utilization of engineered MSC exosomes as a method to deliver both zinc finger activator proteins and mRNAs to increase CFTR expression and demonstrate that that this method can be used as a genetic therapy to therapeutically bolster CFTR expression in those patients that do not respond to the current therapies or as an adjunctive cellular therapeutic approach to treating CF.

Materials and Methods

Cell culture. The Human basal bronchial epithelial cells (HuBEC) from healthy volunteers or CF patients were obtained from bronchoalveolar lavage fluid (BALF) of donors during bronchoscopy. The participants' provided written informed consent. The sample collection was approved by the Sydney Children's Hospital Ethics Review Board (HREC/16/SCHN/120). Basal bronchial cells were conditionally reprogrammed using F-medium, Rock inhibitor and irradiated NIH3T3s feeders based on a previously published protocol (37). At confluency, cells were dissociated using a differential trypsin method and cryopreserved. The HuBEC WT genotype: CFTR wt/wt (normal)), HuBEC CF patients (genotype: CFTR F508del/F508del (CF)) were maintained in Pneumocult™ EX Plus medium (Stemcell Technologies) at 37° C. and 5% CO₂.

Exosomes Isolation and characterization. Human bone marrow-derived mesenchymal stem cells (hBM-MSCs) were obtained from STEMCELL Technologies™. BM-MSCs were expanded in serum-free, conditions (MesenCult®-ACF-XF Attachment Substrate, STEMCELL Technologies) according to the manufacturer's instructions. HEK 293 cells were cultured in Dulbecco Minimum Essential Medium (DMEM) (Mediatech, Manassas, VA) supplemented with exosome depleted serum (Gibco, USA), 50 μg/ml Pen/Strep (Mediatech, Houston, Texas, USA) at 37° C. and 5% CO₂. Six million cells were co-transfected with 15 μg of pCDNA3.1-CFZF-VPR and 5 μg of pDB68 encoding Cx43 S368A, a constitutively active mutant of Cx43 (PhCMV Cx43S368A-pA)(38), plasmids using VIROMER®. The resultant supernatant was collected after 48 hrs from HEK293 cells and 96 hours for MSCs and centrifuged at 800 g for 5 minutes, followed for an additional spin at 2000 g for 10 minutes, supernatant was filtered (0.2 μm) and the ultracentrifuge 100,000 g for 2 hours at 4° C. Pelleted exosomes were re-suspended in PBS and storage at −80° C.

Western Blot. Detection of exosomal protein markers was performed using Western Blot. Exosomes and cells were lysed using RIPA buffer (hermoFisher, USA). 5e+13 particles of extracellular vesicles (EVs) (80-100 μg protein) or/and whole-cell protein (20-50 μg) extract were boiled in 4×laemmli sample buffer (Bio-rad, USA) and loaded onto 4-15% Mini-PROTEAN TGX™ gels (Bio-Rad, USA). Following electrophoresis (100V, 30 mA), the proteins were transferred to a PVDF membrane. The membranes were blocked with 5% non-fat dry milk in TBST and then incubated overnight with primary antibodies CD9 (sc-13118, Santa Cruz, USA), TSG101 (sc-136111, Santa Cruz, USA), CD63 (sc-5275, Santa cruz, USA), Connexin 43 (3D8A5, Invitrogen, USA), Beta Actin (MA1-140, Invitrogen, USA), Anti-Flag M2 (F3165, Sigma, USA). After washing, the membrane was incubated with horseradish peroxidase-conjugated Rabbit anti-mouse IgG and then subjected to enhanced chemiluminescence using super signal West Pico HRP substrate (Thermo Scientific, USA).

Identification of particles by nanoparticle tracking analysis. Nanoparticle tracking analysis (NTA) measurements were performed by using a NanoSight NS300 instrument. The capture settings were Camera Type: sCMOS, Laser Type: Blue488, Camera Level: 9, Slider Shutter: 607, Slider Gain: 15, FPS 25.0, Number of Frames: 1498, Temperature: 23.1-23.1° C., Dilution factor: 1000 and Syringe Pump Speed: 30. The size distribution and quantification of exosome preparations were analyzed by measuring the rate of Brownian motion with a NanoSight LM10 system (NanoSight, Wiltshire United Kingdom) equipped with fast video capture and particle-tracking software NTA 3.3—Sample Assistant Dev Build 3.3.302. Isolated exosomes were diluted (1:1000) and injected into a NanoSight sample cubicle. The mean±SD size distribution of exosomes was determined as well as the mean number of particles per milliliter. For mesenchymal stem cell derived exosomes concentration was: 5.94e+11 particles/ml.

Negative staining electron microscopy of extracellular vesicles. Specimens at certain concentrations were adsorbed to glow-discharged, carbon-coated 200 meshEM copper grids. Samples were prepared by conventional negative staining with 1% (w/v) uranyl acetate. EM images were collected with an FEI Tecnai 12 transmission electron microscope (Thermo Fisher Scientific, Waltham, MA, USA) equipped with a LaB6 filament and operated at an acceleration voltage of 120 kV. Images were recorded with a Gatan 2×2 k CCD camera (Gatan, Inc., Pleasanton, CA, USA) at a magnification of 100 μm and a defocus value of about 1.5 μm.

Exosome labeling. Exosomal membrane was labeled with BODIPY TR ceramide, according to the manufacturer's protocol (Molecular Probes/Invitrogen Life Technologies, Carlsbad, California, USA). Briefly, 5e+10 exosomes were stained with 10 μmol/L BODIPY TR ceramide. Excess fluorescent dye was removed by using Exosome Spin Columns (Life Technologies, Carlsbad, California, USA). BODIPY-labeled exosomes were dropped on 10e+3 cells and incubated in exosome depleted medium at 37° C. and 5% CO₂. Bodipy-labeled exosomes uptake was evaluated by light microscopy.

Construction of CFZP-VPR expression plasmids. The cloning was performed as described before (41). Briefly, a Zinc Finger directed to the CFTR promoter was designed using the zinc finger tools Software™, ordered as a gBlock (IDT, USA) and sub-cloned into the pcDNA3.1 plasmid encoding a ZFP-362-VPR using the NEBuilder® HiFi DNA assembly method to replace the ZFP-362 with ZFP directed to CFTR promoter (CFZF-VPR). HuBEC were transfected with 2 μg of CFZF-VPR plasmid using VIROMER® Red according to the manufacturer's instructions and cultured for 48 hours.

qRT-PCR analysis of gene expression. To determine transcript levels of CFTR total RNA was isolated 48 hours post-transfection using the Maxwell 16 LEV simplyRNA purification kit and the Maxwell 16 Research Instrument (Promega, Madison, WI). DNase-treated RNA samples were then standardized and reverse transcribed with QuantiTect (Qiagen) using an oligo-dT/random monamer primer mix. Quantitative real-time polymerase chain reaction (qRT-PCR) was carried out using Kapa Sybr Fast universal qPCR mix (Kapa Biosystems, Wilmington, MA) on an Eppendorf Mastercycler realplex. Thermal cycling parameters started with 3 minutes at 95° C., followed by 40 cycles of 95° C. for 3 seconds and 60° C. for 30 seconds. The fold change in gene expression was calculated using the 2^{-ΔΔCt} method. Following the RT step, qRT-PCR was carried out with CFTR and β-Actin primers (Table 1).

TABLE 1
Primers sequences

| Name | |
|---|---|
| CFTR F | SEQ ID NO: 21 |
| CFTR R | SEQ ID NO: 22 |
| Beta Actin F | SEQ ID NO: 23 |
| Beta Actin R | SEQ ID NO: 24 |
| ZFA S1F | SEQ ID NO: 25 |
| ZFA S1R | SEQ ID NO: 26 |
| ZFA S2F | SEQ ID NO: 27 |
| ZFA S2R | SEQ ID NO: 28 |
| CFTR Promoter F | SEQ ID NO: 29 |
| CFTR Promoter R | SEQ ID NO: 30 |
| ZFP target CFTR P | SEQ ID NO: 1 |
| Zinc Finger Protein Amino acid Sequence | SEQ ID NO: 32 |
| VPR (protein amino acid sequence) | SEQ ID NO: 33 |

Chromatin immunoprecipitation assay (23). ChIP was performed as described before (39). Briefly, using 5×10⁶ A549 cells transfected with 5 μg of pCDNA-CFZP-VPR, after 48 hours cells were cross-linked, and nuclei was digested using microccocal nuclease. 10% of the nuclei was set aside for input and the rest was incubated in 10 μg of anti-FLAG antibody overnight at 4° C. Thereafter, 50 μl of magnetic protein A/G beads were added and incubated for 2 hours. Samples were eluted reverse crosslinked at 65° C. and treated with RNase and proteinase K. Then, DNA was isolated (ZYMO, USA) and used to perform qPCR. The calculated concentrations were used to determine the enrichment of the protein at the target DNA site, as a fraction of input.

Halide assay. Stable cells expressing EYFP were obtained by transducing CFPAC with pLenti-EYFP plasmid and single cell sorted to select an EYFP cell clone. Human nasal cells WT and F508del expressing transiently EYFP by delivery of Premo™ Halide Sensor (Thermo Fisher Scientific, Waltham, MA, USA) by BacMam technology—Pharmacologically relevant—known modulators show dose-dependent quenching and BacMam delivery enables assays in primary cells. The assay combines the YFP Venus halide sensor with a surrogate ion for chloride (iodide); upon stimulation of the chloride channel or transporter, iodide ions flow down the concentration gradient into the cells and quench YFP fluorescence upon binding; the amount of quench is directly proportional to the ion flux (chloride channel or transporter activity). Cells cultured on 96-well plates were treated with Forskolin, VX809, VX770, amilirate and niflumic acid assayed after using Halide stimulus buffer (NaI 25 mM) and fluorescence evaluated in a plate reader (GloMax®-Promega, Madison, WI, USA). Normalization for expression levels was performed by baseline correction (F/F0).

Light microscopy and imaging. Cells were cultivated fixed with BD cytofix/Cytoperm™ fixation/permeabilization Kit (Cat. No. 554715 BD bioscience, Franklin Lakes, NJ, USA) at room temperature for 20 minutes and then stained with fluorescent 488-Phalloidin (green) for actin using the manufacturer's recommendations (Molecular Probes/Invitrogen Life Technologies). Fluorescence images were collected on Zeiss LSM-700 confocal microscope. For the analysis of the cellular internalization of exosomes Zeiss acquisition parameters, including exposure, focus, illumination, and Z stack projection, were controlled by Zen 2012 Imaging Software for Acquisition and Analysis.

Enzyme-linked Immunosorbent Assay (40). Quantitative detection of human CFTR (Novus Biologicals Centennial, Co, USA) was performed according to the manufacturer's protocol as described previously (47, 48). Briefly, this sandwich ELISA utilizes a microplate coated with an antibody specific to human CFTR. Standards (100 μl) were added in duplicate to the plate. Nasal WT and F508del differentially treated cells were freeze-thaw lysed and samples lysates (100 μl) were added in triplicate to the plate. Next, the plate incubated for 90 minutes at 37° C. in the dark. After incubation the liquid was removed and 100 μl of biotinylated detection antibody working solution added to each well. The plate was covered with the sealer, gently mixed and incubated for 1 hour at 37° C. The solution was aspired from each well and 350 μl of wash buffer added to each well and the plate incubated for 2 minutes at room temperature. Next the solution aspirated from each well followed by 3 washes after which 100 μl of HRP conjugate working solution was added to each well. The HRP exposed plate as was then covered with the plate sealer and incubated for 30 minutes at 37° C. after which the solution was aspirated from each well and the wash step repeated 5 times after which 90 μl of substrate reagent was added to each well; the plate was covered with a new plate sealer and incubated for 15 min at 37° C. in the dark followed by the addition of 50 μl of stop solution added to each well. The optical density (OD value) was determined for each well with the micro-plate reader set to 450 nm. Specification of the kit: Sensitivity: 0.10 ng/ml, Detection Range: 0.16-10 ng/ml.

Statistical analysis. The results are presented as mean of standard error (SEM). For comparison of two groups, the unpaired Student's t-test was used as analyzed in Excel. Differences were considered statistically significant when P<0.05.

Informal Sequence Listing
SEQ ID NO: 1 = ZFP target CFTR P
GGGATGGGCCTGGTGCTGGGC
SEQ ID NO: 2
MDYKDDDDKKRPAATKKAGQAKKKKLEPGEKPYKCPECGK
SFSSKKALTEHQRTHTGEKPYKCPECGKSFSRADNLTEHQ
RTHTGEKPYKCPECGKSFSRSDHLTNHQRTHTGEKPYKCP
ECGKSFSDKKDLTRHQRTHTGEKPYKCPECGKSFSRSDKL
TEHQRTHTGEKPYKCPECGKSFSDPGHLVRHQRTHTGKKT
SSAADPKKKRKVSPGIRRLDALISTSLYKKAGYKEASGSG
RADALDDFDLDMLGSDALDDFDLDMLGSDALDDFDLDMLG
SDALDDFDLDMLINSRSSGSPKKKRKVGSQYLPDTDDRHR
IEEKRKRTYETFKSIMKKSPFSGPTDPRPPPRRIAVPSRS
SASVPKPAPQPYPFTSSLSTINYDEFPTMVFPSGQISQAS
ALAPAPPQVLPQAPAPAPAPAMVSALAQAPAPVPVLAPGP
PQAVAPPAPKPTQAGEGTLSEALLQLQFDDEDLGALLGNS
TDPAVFTDLASVDNSEFQQLLNQGIPVAPHTTEPMLMEYP
EAITRLVTGAQRPPDPAPAPLGAPGLPNGLLSGDEDFSSI
ADMDFSALLGSGSGSRDSREGMFLPKPEAGSAISDVFEGR
EVCQPKRIRPFHPPGSPWANRPLPASLAPTPTGPVHEPVG
SLTPAPVPQPLDPAPAVTPEASHLLEDPDEETSQAVKALR
EMADTVIPQKEEAAICGQMDLSHPPPRGHLDELTTTLESM
TEDLNLDSPLTPELNEILDTFLNDECLLHAMHISTGLSIF
DTSLF
SEQ ID NO: 3
MKRPAATKKAGQAKKKKLEPGEKPYKCPECGKSFSSKKAL
TEHQRTHTGEKPYKCPECGKSFSRADNLTEHQRTHTGEKP
YKCPECGKSFSRSDHLTNHQRTHTGEKPYKCPECGKSFSD
KKDLTRHQRTHTGEKPYKCPECGKSFSRSDKLTEHQRTHT
GEKPYKCPECGKSFSDPGHLVRHQRTHTGKKTSSAADPKK
KRKVSPGIRRLDALISTSLYKKAGYKEASGSGRADALDDF
DLDMLGSDALDDFDLDMLGSDALDDFDLDMLGSDALDDFD
LDMLINSRSSGSPKKKRKVGSQYLPDTDDRHRIEEKRKRT
YETFKSIMKKSPFSGPTDPRPPPRRIAVPSRSSASVPKPA
PQPYPFTSSLSTINYDEFPTMVFPSGQISQASALAPAPPQ
VLPQAPAPAPAPAMVSALAQAPAPVPVLAPGPPQAVAPPA
PKPTQAGEGTLSEALLQLQFDDEDLGALLGNSTDPAVFTD
LASVDNSEFQQLLNQGIPVAPHTTEPMLMEYPEAITRLVT
GAQRPPDPAPAPLGAPGLPNGLLSGDEDFSSIADMDFSAL
LGSGSGSRDSREGMFLPKPEAGSAISDVFEGREVCQPKRI
RPFHPPGSPWANRPLPASLAPTPTGPVHEPVGSLTPAPVP
QPLDPAPAVTPEASHLLEDPDEETSQAVKALREMADTVIP
QKEEAAICGQMDLSHPPPRGHLDELTTTLESMTEDLNLDS
PLTPELNEILDTFLNDECLLHAMHISTGLSIFDTSLF
SEQ ID NO: 4
MLEPGEKPYKCPECGKSFSSKKALTEHQRTHTGEKPYKCP
ECGKSFSRADNLTEHQRTHTGEKPYKCPECGKSFSRSDHL
TNHQRTHTGEKPYKCPECGKSFSDKKDLTRHQRTHTGEKP
YKCPECGKSFSRSDKLTEHQRTHTGEKPYKCPECGKSFSD
PGHLVRHQRTHTGKKTSSAADPKKKRKVSPGIRRLDALIS
TSLYKKAGYKEASGSGRADALDDFDLDMLGSDALDDFDLD
MLGSDALDDFDLDMLGSDALDDFDLDMLINSRSSGSPKKK
RKVGSQYLPDTDDRHRIEEKRKRTYETFKSIMKKSPFSGP
TDPRPPPRRIAVPSRSSASVPKPAPQPYPFTSSLSTINYD
EFPTMVFPSGQISQASALAPAPPQVLPQAPAPAPAPAMVS
ALAQAPAPVPVLAPGPPQAVAPPAPKPTQAGEGTLSEALL
QLQFDDEDLGALLGNSTDPAVFTDLASVDNSEFQQLLNQG
IPVAPHTTEPMLMEYPEAITRLVTGAQRPPDPAPAPLGAP
GLPNGLLSGDEDFSSIADMDFSALLGSGSGSRDSREGMFL
PKPEAGSAISDVFEGREVCQPKRIRPFHPPGSPWANRPLP
ASLAPTPTGPVHEPVGSLTPAPVPQPLDPAPAVTPEASHL
LEDPDEETSQAVKALREMADTVIPQKEEAAICGQMDLSHP
PPRGHLDELTTTLESMTEDLNLDSPLTPELNEILDTFLND
ECLLHAMHISTGLSIFDTSLF
SEQ ID NO: 5
MKRPAATKKAGQAKKKKLEPGEKPYKCPECGKSFSSKKAL
TEHQRTHTGEKPYKCPECGKSFSRADNLTEHQRTHTGEKP
YKCPECGKSFSRSDHLTNHQRTHTGEKPYKCPECGKSFSD
KKDLTRHQRTHTGEKPYKCPECGKSFSRSDKLTEHQRTHT
GEKPYKCPECGKSFSDPGHLVRHQRTHTGKKTSSPKKKRK
VSGSGDALDDFDLDMLGSDALDDFDLDMLGSDALDDFDLD
MLGSDALDDFDLDMLSSGSPKKKRKVGSQYLPDTDDRHRI
EEKRKRTYETFKSIMKKSPFSGPTDPRPPPRRIAVPSRSS
ASVPKPAPQPYPFTSSLSTINYDEFPTMVFPSGQISQASA
LAPAPPQVLPQAPAPAPAPAMVSALAQAPAPVPVLAPGPP
QAVAPPAPKPTQAGEGTLSEALLQLQFDDEDLGALLGNST
DPAVFTDLASVDNSEFQQLLNQGIPVAPHTTEPMLMEYPE
AITRLVTGAQRPPDPAPAPLGAPGLPNGLLSGDEDFSSIA
DMDFSALLGSGSGSRDSREGMFLPKPEAGSAISDVFEGRE
VCQPKRIRPFHPPGSPWANRPLPASLAPTPTGPVHEPVGS
LTPAPVPQPLDPAPAVTPEASHLLEDPDEETSQAVKALRE
MADTVIPQKEEAAICGQMDLSHPPPRGHLDELTTTLESMT
EDLNLDSPLTPELNEILDTFLNDECLLHAMHISTGLSIFD
TSLF
SEQ ID NO: 6
MLEPGEKPYKCPECGKSFSSKKALTEHQRTHTGEKPYKCP
ECGKSFSRADNLTEHQRTHTGEKPYKCPECGKSFSRSDHL
TNHQRTHTGEKPYKCPECGKSFSDKKDLTRHQRTHTGEKP
YKCPECGKSFSRSDKLTEHQRTHTGEKPYKCPECGKSFSD
PGHLVRHQRTHTGKKTSSPKKKRKVSGSGDALDDFDLDML
GSDALDDFDLDMLGSDALDDFDLDMLGSDALDDFDLDMLS
SGSPKKKRKVGSQYLPDTDDRHRIEEKRKRTYETFKSIMK
KSPFSGPTDPRPPPRRIAVPSRSSASVPKPAPQPYPFTSS
LSTINYDEFPTMVFPSGQISQASALAPAPPQVLPQAPAPA
PAPAMVSALAQAPAPVPVLAPGPPQAVAPPAPKPTQAGEG
TLSEALLQLQFDDEDLGALLGNSTDPAVFTDLASVDNSEF
QQLLNQGIPVAPHTTEPMLMEYPEAITRLVTGAQRPPDPA
PAPLGAPGLPNGLLSGDEDFSSIADMDFSALLGSGSGSRD
SREGMFLPKPEAGSAISDVFEGREVCQPKRIRPFHPPGSP
WANRPLPASLAPTPTGPVHEPVGSLTPAPVPQPLDPAPAV
TPEASHLLEDPDEETSQAVKALREMADTVIPQKEEAAICG
QMDLSHPPPRGHLDELTTTLESMTEDLNLDSPLTPELNEI
LDTFLNDECLLHAMHISTGLSIFDTSLF
SEQ ID NO: 7
LEPGEKPYKCPECGKSFSSKKALTEHQRTHTGEKPYKCPE
CGKSFSRADNLTEHQRTHTGEKPYKCPECGKSFSRSDHLT
NHQRTHTGEKPYKCPECGKSFSDKKDLTRHQRTHTGEKPY
KCPECGKSFSRSDKLTEHQRTHTGEKPYKCPECGKSFSDP
GHLVRHQRTHTGKKTSSDALDDFDLDMLGSDALDDFDLDM
LGSDALDDFDLDMLGSDALDDFDLDMLQYLPDTDDRHRIE
EKRKRTYETFKSIMKKSPFSGPTDPRPPPRRIAVPSRSSA
SVPKPAPQPYPFTSSLSTINYDEFPTMVFPSGQISQASAL
APAPPQVLPQAPAPAPAPAMVSALAQAPAPVPVLAPGPPQ
AVAPPAPKPTQAGEGTLSEALLQLQFDDEDLGALLGNSTD
PAVFTDLASVDNSEFQQLLNQGIPVAPHTTEPMLMEYPEA
ITRLVTGAQRPPDPAPAPLGAPGLPNGLLSGDEDFSSIAD
MDFSALLRDSREGMFLPKPEAGSAISDVFEGREVCQPKRI
RPFHPPGSPWANRPLPASLAPTPTGPVHEPVGSLTPAPVP
QPLDPAPAVTPEASHLLEDPDEETSQAVKALREMADTVIP
QKEEAAICGQMDLSHPPPRGHLDELTTTLESMTEDLNLDS
PLTPELNEILDTFLNDECLLHAMHISTGLSIFDTSLF
SEQ ID NO: 8
LEPGEKPYKCPECGKSFSSKKALTEHQRTHTGEKPYKCPE
CGKSFSRADNLTEHQRTHTGEKPYKCPECGKSFSRSDHLT
NHQRTHTGEKPYKCPECGKSFSDKKDLTRHQRTHTGEKPY
KCPECGKSFSRSDKLTEHQRTHTGEKPYKCPECGKSFSDP
GHLVRHQRTHTGKKTSS
SEQ ID NO: 9
DALDDFDLDMLGSDALDDFDLDMLGSDALDDFDLDMLGSD
ALDDFDLDML
SEQ ID NO: 10
QYLPDTDDRHRIEEKRKRTYETFKSIMKKSPFSGPTDPRP
PPRRIAVPSRSSASVPKPAPQPYPFTSSLSTINYDEFPTM
VFPSGQISQASALAPAPPQVLPQAPAPAPAPAMVSALAQA
PAPVPVLAPGPPQAVAPPAPKPTQAGEGTLSEALLQLQFD
DEDLGALLGNSTDPAVFTDLASVDNSEFQQLLNQGIPVAP
HTTEPMLMEYPEAITRLVTGAQRPPDPAPAPLGAPGLPNG
LLSGDEDFSSIADMDFSALL
SEQ ID NO: 11
RDSREGMFLPKPEAGSAISDVFEGREVCQPKRIRPFHPPG
SPWANRPLPASLAPTPTGPVHEPVGSLTPAPVPQPLDPAP
AVTPEASHLLEDPDEETSQAVKALREMADTVIPQKEEAAI
CGQMDLSHPPPRGHLDELTTTLESMTEDLNLDSPLTPELN
EILDTFLNDECLLHAMHISTGLSIFDTSLF
SEQ ID NO: 12 = NLS
KRPAATKKAGQAKKKK
SEQ ID NO: 13 = NLS
PKKKRKV
SEQ ID NO: 14 = Cell-Penetrating Peptide
GRKKRRQRRR
SEQ ID NO: 15 = Cell-Penetrating Peptide
GRKKRRQRRRVDL
SEQ ID NO: 16 = cMyc tag peptide
EQKLISEEDLL
SEQ ID NO: 17 = FLAG-Tag
DYKDDDDK
SEQ ID NO: 18 = Connexin43
MGDWSALGKLLDKVQAYSTAGGKVWLSVLFIFRILLLGTA
VESAWGDEQSAFRCNTQQPGCENVCYDKSFPISHVRFWVL
QIIFVSVPTLLYLAHVFYVMRKEEKLNKKEEELKVAQTDG
VNVDMHLKQIEIKKFKYGIEEHGKVKMRGGLLRTYIISIL
FKSIFEVAFLLIQWYIYGFSLSAVYTCKRDPCPHQVDCFL
SRPTEKTIFIIFMLVVSLVSLALNIIELFYVFFKGVKDRV
KGKSDPYHATSGALSPAKDCGSQKYAYFNGCSSPTAPLSP
MSPPGYKLVTGDRNNSSCRNYNKQASEQNWANYSAEQNRM
GQAGSTISNSHAQPFDFPDDNQNSKKLAAGHELQPLAIVD
QRPSSRASSRASSRPRPDDLEI
SEQ ID NO: 19 = Connexin43
MGDWSALGKLLDKVQAYSTAGGKVWLSVLFIFRILLLGTA
VESAWGDEQSAFRCNTQQPGCENVCYDKSFPISHVRFWVL
QIIFVSVPTLLYLAHVFYVMRKEEKLNKKEEELKVAQTDG
VNVDMHLKQIEIKKFKYGIEEHGKVKMRGGLLRTYIISIL
FKSIFEVAFLLIQWYIYGFSLSAVYTCKRDPCPHQVDCFL
SRPTEKTIFIIFMLVVSLVSLALNIIELFYVFFKGVKDRV
KGKSDPYHATSGALSPAKDCGSQKYAYFNGCSSPTAPLSP
MSPPGYKLVTGDRNNSSCRNYNKQASEQNWANYSAEQNRM
GQAGSTISNSHAQPFDFPDDNQNSKKLAAGHELQPLAIVD
QRPSSRAASRASSRPRPDDLEI
SEQ ID NO: 20
MLGSDALDDFDLDMLINSRSSGSPKKKRKVGSQYLPDTDD
RHRIEEKRKRTYETFKSIMKKSPFSGPTDPRPPPRRIAVP
SRSSASVPKPAPQPYPFTSSLSTINYDEFPTMVFPSGQIS
QASALAPAPPQVLPQAPAPAPAPAMVSALAQAPAPVPVLA
PGPPQAVAPPAPKPTQAGEGTLSEALLQLQFDDEDLGALL
GNSTDPAVFTDLASVDNSEFQQLLNQGIPVAPHTTEPMLM
EYPEAITRLVTGAQRPPDPAPAPLGAPGLPNGLLSGDEDF
SSIADMDFSALLGSGSGSRDSREGMFLPKPEAGSAISDVF
EGREVCQPKRIRPFHPPGSPWANRPLPASLAPTPTGPVHE
PVGSLTPAPVPQPLDPAPAVTPEASHLLEDPDEETSQAVK
ALREMADTVIPQKEEAAICGQMDLSHPPPRGHLDELTTTL
ESMTEDLNLDSPLTPELNEILDTFLNDECLLHAMHISTGL
SIFDTSLF
SEQ ID NO: 21 = CTCATGGGATGTGATTCTTTCGACC
SEQ ID NO: 22 = TTTCTGTCCAGGAGACAGGAGCAT
SEQ ID NO: 23 = CACCAACTGGGACGACAT
SEQ ID NO: 24 = ACAGCCTGGATAGCAACG
SEQ ID NO: 25 = GGACTATAAGGACGACGATGACAAGA
SEQ ID NO: 26 = GTATGGGTCCTCTGATGGCG
SEQ ID NO: 27 = TGTCCCGAATGCGGCA
SEQ ID NO: 28 = AGGGCGTCACTGCCG
SEQ ID NO: 29 = CTGGGTCTGGCGGACCCTGA
SEQ ID NO: 30 = TCGCGCGCGCTCCTTCCAGG
SEQ ID NO: 31
ATGGACTATAAGGACGACGATGACAAGAAGAGGCCAGCGG
CTACTAAAAAGGCTGGACAGGCCAAAAAGAAAAAACTGGA
GCCCGGCGAAAAGCCGTATAAGTGCCCAGAATGTGGGAAG
AGTTTTTCTAGTAAGAAAGCATTGACGGAGCACCAGAGAA
CACACACAGGTGAAAAGCCTTACAAATGCCCTGAATGCGG
TAAAAGCTTCAGTCGAGCGGATAATCTCACGGAGCATCAA
CGGACACACACTGGTGAAAAACCTTATAAATGTCCCGAAT
GCGGCAAATCTTTCAGTAGAAGTGATCATCTTACTAACCA
CCAGCGCACCCACACAGGCGAGAAACCGTATAAATGCCCC
GAGTGTGGTAAGTCCTTTTCCGATAAAAAAGATCTTACTC
GCCACCAACGCACCCACACGGGAGAAAAACCATATAAGTG
TCCTGAGTGTGGCAAATCTTTTTCTAGGAGTGATAAACTT
ACGGAACACCAGAGGACTCATACTGGAGAGAAGCCATATA
AATGTCCGGAATGCGGGAAAAGTTTTTCAGATCCAGGTCA
CTTGGTGCGCCATCAGAGGACCCATACGGGAAAAAAGACG
AGCAGCGCTGCTGACCCCAAGAAGAAGAGGAAGGTGTCGC
CAGGGATCCGTCGACTTGACGCGTTGATATCAACAAGTTT
GTACAAAAAAGCAGGCTACAAAGAGGCCAGCGGTTCCGGA
CGGGCTGACGCATTGGACGATTTTGATCTGGATATGCTGG
GAAGTGACGCCCTCGATGATTTTGACCTTGACATGCTTGG
TTCGGATGCCCTTGATGACTTTGACCTCGACATGCTCGGC
AGTGACGCCCTTGATGATTTCGACCTGGACATGCTGATTA
ACTCTAGAAGTTCCGGATCTCCGAAAAAGAAACGCAAAGT
TGGTAGCCAGTACCTGCCCGACACCGACGACCGGCACCGG
ATCGAGGAAAAGCGGAAGCGGACCTACGAGACATTCAAGA
GCATCATGAAGAAGTCCCCCTTCAGCGGCCCCACCGACCC
TAGACCTCCACCTAGAAGAATCGCCGTGCCCAGCAGATCC
AGCGCCAGCGTGCCAAAACCTGCCCCCCAGCCTTACCCCT
TCACCAGCAGCCTGAGCACCATCAACTACGACGAGTTCCC
TACCATGGTGTTCCCCAGCGGCCAGATCTCTCAGGCCTCT
GCTCTGGCTCCAGCCCCTCCTCAGGTGCTGCCTCAGGCTC
CTGCTCCTGCACCAGCTCCAGCCATGGTGTCTGCACTGGC
TCAGGCACCAGCACCCGTGCCTGTGCTGGCTCCTGGACCT
CCACAGGCTGTGGCTCCACCAGCCCCTAAACCTACACAGG
CCGGCGAGGGCACACTGTCTGAAGCTCTGCTGCAGCTGCA
GTTCGACGACGAGGATCTGGGAGCCCTGCTGGGAAACAGC
ACCGATCCTGCCGTGTTCACCGACCTGGCCAGCGTGGACA
ACAGCGAGTTCCAGCAGCTGCTGAACCAGGGCATCCCTGT
GGCCCCTCACACCACCGAGCCCATGCTGATGGAATACCCC
GAGGCCATCACCCGGCTCGTGACAGGCGCTCAGAGGCCTC
CTGATCCAGCTCCTGCCCCTCTGGGAGCACCAGGCCTGCC
TAATGGACTGCTGTCTGGCGACGAGGACTTCAGCTCTATC
GCCGATATGGATTTCTCAGCCTTGCTGGGCTCTGGCAGCG
GCAGCCGGGATTCCAGGGAAGGGATGTTTTTGCCGAAGCC
TGAGGCCGGCTCCGCTATTAGTGACGTGTTTGAGGGCCGC
GAGGTGTGCCAGCCAAAACGAATCCGGCCATTTCATCCTC
CAGGAAGTCCATGGGCCAACCGCCCACTCCCCGCCAGCCT
CGCACCAACACCAACCGGTCCAGTACATGAGCCAGTCGGG
TCACTGACCCCGGCACCAGTCCCTCAGCCACTGGATCCAG
CGCCCGCAGTGACTCCCGAGGCCAGTCACCTGTTGGAGGA
TCCCGATGAAGAGACGAGCCAGGCTGTCAAAGCCCTTCGG
GAGATGGCCGATACTGTGATTCCCCAGAAGGAAGAGGCTG
CAATCTGTGGCCAAATGGACCTTTCCCATCCGCCCCCAAG
GGGCCATCTGGATGAGCTGACAACCACACTTGAGTCCATG
ACCGAGGATCTGAACCTGGACTCACCCCTGACCCCGGAAT
TGAACGAGATTCTGGATACCTTCCTGAACGACGAGTGCCT
CTTGCATGCCATGCATATCAGCACAGGACTGTCCATCTTC
GACACATCTCTGTTTTGA
SEQ ID NO: 32
LEPGEKPYKCPECGKSFSDPGHLVRHQRTHTGEKPYKCPE
CGKSFSRNDALTEHQRTHTGEKPYKCPECGKSFSRSDELV
RHQRTHTGEKPYKCPECGKSFSRNDALTEHQRTHTGEKPY
KCPECGKSFSDPGHLVRHQRTHTGEKPYKCPECGKSFSRR
DELNVHQRTHTGEKPYKCPECGKSFSRSDKLVRHQRTHTG
KKTS
SEQ ID NO: 33
MLGSDALDDFDLDMLGSDALDDFDLDMLGSDALDDFDLDM
LINSRSSGSPKKKRKVGSQYLPDTDDRHRIEEKRKRTYET
FKSIMKKSPFSGPTDPRPPPRRIAVPSRSSASVPKPAPQP
YPFTSSLSTINYDEFPTMVFPSGQISQASALAPAPPQVLP
QAPAPAPAPAMVSALAQAPAPVPVLAPGPPQAVAPPAPKP
TQAGEGTLSEALLQLQFDDEDLGALLGNSTDPAVFTDLAS
VDNSEFQQLLNQGIPVAPHTTEPMLMEYPEAITRLVTGAQ
RPPDPAPAPLGAPGLPNGLLSGDEDFSSIADMDFSALLGS
GSGSRDSREGMFLPKPEAGSAISDVFEGREVCQPKRIRPF
HPPGSPWANRPLPASLAPTPTGPVHEPVGSLTPAPVPQPL
DPAPAVTPEASHLLEDPDEETSQAVKALREMADTVIPQKE
EAAICGQMDLSHPPPRGHLDELTTTLESMTEDLNLDSPLT
PELNEILDTFLNDECLLHAMHISTGLSIFDTSLF

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Riordan et al. Science. 1989; 245(4922):1066-73.
2. Sheppard et al Physiol Rev. 1999; 79(1 Suppl):S23-45.
3. Bobadilla et al Hum Mutat. 2002; 19(6):575-606.
4. Du et al Nature structural & molecular biology. 2005; 12(1):17-25.
5. Thomas et al FEBS letters. 1992; 312(1):7-9.
6. Kim et al. Front Pharmacol. 2012; 3:201. PMCID: 3521238.
7. Romey et al. Ann Biol Clin (Paris). 2006; 64(5):429-37.
8. Lopes-Pacheco et al. Front Pharmacol. 2016; 7:275. PMCID: 5011145.
9. Chaudary et al. Ther Clin Risk Manag. 2018; 14:2375-83. PMCID: 6287538.
10. Van Goor et al. Proc Natl Acad Sci USA. 2011; 108(46):18843-8. PMCID: 3219147.
11. Mandell et al. Nucleic Acids Res. 2006; 34(Web Server issue):W516-23. PMCID: 1538883.
12. Graslund et al. J Biol Chem. 2005; 280(5):3707-14.
13. Scott T S, D. O'meally, N. A. Grepo, M. S. Weinberg, V. Planelles, and K V. Morris. Broadly active zinc finger protein-guided transcriptional activation of HIV-1. Molecular Therapy Methods & Clinical Development. 2020.
14. Fevrier et al. Curr Opin Cell Biol. 2004; 16(4):415-21.
15. Turturici et al. Am J Physiol Cell Physiol. 2014; 306(7):C621-33.
16. Desrochers eta 1. Dev Cell. 2016; 37(4):301-9. PMCID: 4995598.
17. Pegtel et al. Annu Rev Biochem. 2019; 88:487-514.
18. Valadi et al. Nat Cell Biol. 2007; 9(6):654-9.
19. Bang et al. Int J Biochem Cell Biol. 2012; 44(11):2060-4.
20. Phan et al. J Extracell Vesicles. 2018; 7(1):1522236. PMCID: 6161586.
21. Romanelli et al. Front Neurol. 2019; 10:1225. PMCID: PMC6896947.
22. Lin et al. Front Immunol. 2018; 9:2256. PMCID: PMC6175982.
23. Brena et al. Evol Dev. 2006; 8(3):252-65.
24. O'Brien et al. Nat Rev Mol Cell Biol. 2020; 21(10):585-606. PMCID: PMC7249041.
25. Kojima et al. Nat Commun. 2018; 9(1):1305. PMCID: PMC5880805.
26. Wilber et al. Blood. 2010; 115(15):3033-41. PMCID: 2858469.
27. Perdigao et al. Mol Ther Methods Clin Dev. 2020; 18:145-58. PMCID: 7317221.
28. Popowski et al. J Extracell Vesicles. 2020; 9(1):1785161.
29. Villamizar et al. Targeted Activation of Cystic Fibrosis Transmembrane Conductance Regulator. Mol Ther. 2019; 27(10):1737-48. PMCID: 6822231.
30. Singh et al. Genes (Basel). 2020; 11(4). PMCID: 7230663.
31. Inal et al. Clin Sci (Lond). 2020; 134(12):1301-4. PMCID: 7298154.
32. Liu et al. Theranostics. 2019; 9(4):1015-28. PMCID: 6401399.
33. Janas et al. FEBS Lett. 2015; 589(13):1391-8.
34. Janas et al. FEBS letters. 2015; 589(13):1391-8.
35. Gemel et al. Cancers (Basel). 2019; 11(4). PMCID: 6520873.
36. Soares et al. Sci Rep. 2015; 5:13243. PMCID: 4541155.
37. Martinovich et al. Scientific reports. 2017; 7(1):17971. PMCID: 5740081.
38. Kojima et al. Nat Commun. 2018; 9(1):1305. PMCID: 5880805.
39. Ray R M, Hansen A H, Slott S, Taskova M, Astakhova K, Morris K V. Control of LDL Uptake in Human Cells by Targeting the LDLR Regulatory Long Non-coding RNA BM450697. Mol Ther Nucleic Acids. 2019; 17:264-76. PMCID: PMC6611981.
40. Nesterova et al. Genome Res. 2001; 11(5):833-49.
41. Scott T A, O'Meally D, Grepo N A, Soemardy C, Lazar D C, Zheng Y, Weinberg M S, Planelles V, Morris K V, Broadly active zinc finger protein-guided transcriptional activation of HIV-1, Molecular Therapy: Methods & Clinical Development (2020), doi: https://doi.org/10.1016/j.omtm.2020.10.018

1-81. (canceled)

82. A zinc finger domain having at least 90% sequence identity to SEQ ID NO:8.

83. A fusion protein comprising the zinc finger domain of claim 82, wherein the fusion protein is capable of binding to the promoter sequence of a cystic fibrosis transmembrane conductance regulator gene.

84. The fusion protein of claim 83, wherein the promoter sequence of the cystic fibrosis transmembrane conductance regulator gene has at least 95% sequence identity to SEQ ID NO:1.

85. The fusion protein of claim 83, comprising, from N-terminus to C-terminus, the zinc finger domain, VP64 having at least 90% sequence identity to SEQ ID NO:9, p65 having at least 90% sequence identity to SEQ ID NO:10, and Rta having at least 90% sequence identity to SEQ ID NO:11.

86. The fusion protein of claim 85, further comprising a nuclear localization signal having SEQ ID NO:12 or SEQ ID NO:13; a TAT domain having SEQ ID NO:14 or SEQ ID NO:15; a Myc tag having SEQ ID NO:16, or a combination of two or more thereof.

87. A complex comprising the fusion protein claim 83 bonded to a promoter sequence of a cystic fibrosis transmembrane conductance regulator gene.

88. The complex of claim 87, wherein the cystic fibrosis transmembrane conductance regulator gene has a deletion of a phenylalanine residue at position 508.

89. A nucleic acid encoding the fusion protein of claim 83.

90. The nucleic acid of claim 89, further encoding a connexin 43 peptide.

91. The nucleic acid of claim 90, wherein the connexin 43 peptide comprises SEQ ID NO:18 or SEQ ID NO:19.

92. A vector comprising a nucleic acid encoding the fusion protein of claim 83.

93. The vector of claim 92, further comprising a nucleic acid encoding a connexin 43 peptide.

94. An exosome comprising the fusion protein of claim 83.

95. The exosome of claim 94, further comprising a connexin 43 peptide; a nucleic acid encoding a connexin 43 peptide; or a vector comprising a nucleic acid encoding a connexin 43 peptide.

96. A cell comprising the fusion protein of claim 83.

97. A pharmaceutical composition comprising the fusion protein of claim 83 and a pharmaceutically acceptable excipient.

98. A method of treating cystic fibrosis in a patient in need thereof, the method comprising administering to the patient an effective amount of the fusion protein of claim 83.

99. A method of treating cystic fibrosis in a patient in need thereof, the method comprising:
(i) transducing cells ex vivo with a nucleic acid encoding the fusion protein of claim 83 to produce transduced cells; and
(ii) administering an effective amount of the transduced cells to the patient, thereby treating cystic fibrosis.

100. The method of claim 99, wherein the patient has a deletion of a phenylalanine residue at position 508 in the cystic fibrosis transmembrane conductance regulator gene.

101. A method of activating transcription in a cystic fibrosis transmembrane conductance regulator gene, the method comprising contacting an effective amount of the fusion protein of claim 83 with the cystic fibrosis transmembrane conductance regulator gene.
